(19) United States
(12) Patent Application Publication
(10) Pub. No.: US 2009/0068102 A1
(43) Pub. Date: Mar. 12, 2009

(54) TREATING STROKE

(75) Inventors: Linda C. Burkly, West Newton, MA (US); Kyungmin Hahm, Lexington, MA (US); Marcus Schwaninger, Heildelberg (DE); Ionta Inta, Heidelberg (DE)

Correspondence Address: BIOGEN IDEC / FINNEGAN HENDERSON, LLP, 901 NEW YORK AVENUE, NW, WASHINGTON, DC, 20001-4413, US

(73) Assignees: BIOGEN IDEC MA INC., Cambridge, MA (US); RUPRECHT-KARLS-UNIVERSITAET HEIDELBERG, Heidelberg (DE)

(21) Appl. No.: 11/816,500

(22) PCT Filed: Feb. 15, 2006
(86) PCT No.: PCT/US2006/005217

§ 371 Date: Oct. 16, 2008

Related U.S. Application Data
(60) Provisional application No. 60/653,811, filed on Feb. 17, 2005.

(51) Int. Cl. A61K 51/08; A61K 39/395; A61K 38/17; A61K 39/44; C12Q 1/02; C12Q 1/68

(52) U.S. Cl. 424/1.69; 424/158.1; 514/12; 424/135.1; 424/133.1; 424/178.1; 435/29; 435/6

(57) ABSTRACT

Methods of treating stroke with blocking agents of TWEAK or TWEAK receptor are presented.

### BACKGROUND

Stroke is a leading cause of death and disability worldwide. About 700,000 Americans will have a stroke this year. In the United States, stroke is the third most-frequent cause of death and a leading cause of severe, long-term disability.

### SUMMARY

TWEAK (TNF-like weak inducer of apoptosis) is a trimeric protein that is a TNF (Tumor Necrosis Factor) superfamily member. TWEAK mediates cellular responses by activating a cell surface receptor (referred to as TWEAK receptor or TWEAK-R herein), such as the Fn14 protein. As further described below, blocking TWEAK/TWEAK-R interaction is an effective and useful therapy for stroke.

In one aspect, the disclosure features a method for treating a subject who has had a stroke and/or who is at risk for stroke. The method includes administering a TWEAK/TWEAK-R blocking agent to the subject. A “TWEAK/TWEAK-R blocking agent” or “agent that blocks a TWEAK/TWEAK-R interaction or activity” refers to an agent (e.g., any compound) that at least partially inhibits an interaction or activity of a TWEAK or TWEAK-R. For example, the agent at least partially inhibits an activity, e.g., binding of TWEAK to a TWEAK-R, or the agent at least partially inhibits a nucleic acid encoding TWEAK or TWEAK-R, e.g., to reduce TWEAK or TWEAK-R protein expression.

In one embodiment, the agent reduces the ability of TWEAK to bind to Fn14 (a TWEAK receptor), e.g., reduces affinity of TWEAK-Fn14 binding by a factor of at least 5, 10, 20, 50, or 100. The agent can be an antibody that binds to TWEAK or Fn14. The antibody can be an IgG, e.g., a full length IgG. In one embodiment, the antibody is human, humanized, or effectively human.

In another embodiment, the agent can be a soluble form of a TWEAK receptor, e.g., a human TWEAK receptor such as Fn14. The soluble form of the TWEAK receptor can be fused with a heterologous polypeptide sequence, e.g., a peptide tag or an antibody Fc region.

In one embodiment, the agent can be administered in an amount and/or for a time sufficient to reduce ischemic damage in neuronal tissue in the brain.

The subject is typically a mammal, e.g., human, dog, cat, monkey, rabbit, or agriculture mammal (e.g., horse, cow, pig, and so on). For example, the subject is a human, e.g., a human male or female. The subject can be at least 18, 25, 30, 45, 50, 55, 60, or 70 years old.

In one embodiment, the subject has experienced a stroke. The stroke can be a hemorrhagic stroke, ischemic stroke, or a transient ischemic attack (TIA).

In one embodiment, the subject has experienced a stroke within the last 48 hours, e.g., within the last 2, 3, 5, 8, 12, 20, or 30 hours. In another embodiment, the subject has experienced a stroke more than 48 hours before, but within the last two or three weeks or months.

In another embodiment, the subject is at risk for stroke, e.g., has experienced or is experiencing conditions that create a risk for stroke. Examples of such conditions include high blood pressure; tobacco use; diabetes mellitus; carotid or other artery disease (e.g., peripheral artery disease); atrial fibrillation; other heart disease; transient ischemic attacks (TIAs); certain blood disorders (e.g., high red blood cell count; Sickle cell disease); high blood cholesterol; physical inactivity and obesity; excessive alcohol; some illegal drags; a prior stroke; or prior heart attack.

In one embodiment, the agent is administered in an amount sufficient to reduce infarct size, e.g., by at least 5, 10, 15, 20, 40, 50, 60, 70, or 80%, in neuronal tissue in the brain, relative to the infarct size in an untreated subject. The amount sufficient to reduce infarct size can be evaluated using an animal model, e.g., as described herein.

In one embodiment, the agent is administered in an amount sufficient to improve symptoms in one or more stoke assessment criterion, e.g., a criterion described herein, by at least 5, 10, 15, 20, 40, 50, 60, 70, or 80%.

In one embodiment, the agent is administered in combination with a treatment for stoke. For example, the treatment includes administering another agent that provides a therapeutic benefit to a patient who has or is at risk for stroke, e.g., an agent that is other than a blocking agent of TWEAK and TWEAK receptor interaction or activity, e.g., a thrombolytic agent. In another embodiment, the agent is administered in combination with at least one other TWEAK/TWEAK-R blocking agent described herein.

In one embodiment, the subject exhibits one or more of the following symptoms: sudden numbness or weakness of the face; sudden numbness or weakness of an arm; sudden numbness or weakness of a leg; sudden confusion; sudden trouble speaking; sudden trouble understanding; sudden trouble seeing in one or both eyes; sudden trouble walking; sudden dizziness; sudden loss of balance or coordination; sudden and severe headache with no known cause. In some embodiments, the subject has been diagnosed as having sustained a stroke.

In one embodiment, the method also includes evaluating the subject for a post-stroke criterion. For example, the information from the evaluation can be used to determine whether to continue or discontinue providing a TWEAK/TWEAK-R blocking agent.

The method can include other features described herein.

In another aspect, the method includes a step of identifying a subject who has a stroke (e.g., ischemic stroke, hemorrhagic stroke, or transient ischemic attack) or symptoms of a stoke and administering an agent that blocks a TWEAK/TWEAK-R interaction or activity to the subject. The method can include other features described herein.

In one aspect, the disclosure features an agent that blocks a TWEAK/TWEAK-R interaction or activity for use in treating stroke, e.g., as described herein. The agent can be a blocking agent further described herein. In another aspect, the disclosure features the use of an agent that blocks a TWEAK/TWEAK-R interaction or activity for the manufacture of a medicament for treating stroke, e.g., as described herein. The agent can be a blocking agent further described herein.

In one aspect, the disclosure features a container that includes an agent that blocks a TWEAK/TWEAK-R interaction or activity (e.g., an agent described herein) and a label with instructions for use of the agent in treating stroke.

In another aspect, the disclosure features a method of evaluating a subject. The method includes detecting a TWEAK or TWEAK-R (e.g., Fn14) protein or a nucleic acid encoding TWEAK or TWEAK-R in a subject. In one embodiment, the method includes correlating the result of the detection with the subject's risk for stroke. The term “correlating” refers to describing the relationship between the presence or level of TWEAK or TWEAK-R protein or nucleic acid, and the presence or level of risk for stroke. For example, increased expression can indicate that the subject has had a stroke or is at risk for stoke. Such correlation may be displayed in a record, e.g., a printed or computer readable material, e.g., an informational, diagnostic, or instructional material, e.g., to the subject, health care provider, or insurance company, identifying the presence or level of TWEAK or TWEAK-R protein or nucleic acid as a risk or diagnostic factor for stroke. In another embodiment, increased expression can indicate that the subject has had a stroke. In one embodiment, a labeled agent that binds to TWEAK or TWEAK-R is administered to the subject and the subject is monitored (e.g., scanned) to detect one or more locations in the brain where TWEAK or a TWEAK receptor is expressed. The method can identify locations where TWEAK or TWEAK receptor expression is increased. In other embodiments, TWEAK or TWEAK receptor expression is detected in a biological sample from the subject.

In another aspect, the disclosure features a method of evaluating a subject for a stroke- or stroke-risk assessment. The method includes evaluating TWEAK or TWEAKR protein or a nucleic acid encoding TWEAK or a TWEAK receptor in the subject or in a sample obtained from the subject. For example, the step of evaluating includes evaluating expression or activity of a TWEAK or TWEAK-R protein or a nucleic acid encoding TWEAK or a TWEAK receptor (e.g., by qualitative or quantitative analysis of mRNA, cDNA, or protein), or evaluating one or more nucleotides in a nucleic acid (genomic, mRNA, or cDNA) encoding TWEAK or a TWEAK receptor. In one embodiment, the subject has sustained a stroke or a TIA, or is suspected of having sustained a stroke. In one embodiment, the method includes administering a labeled TWEAK or TWEAK-R binding agent (e.g., an antibody) to a subject, and evaluating localization of the labeled binding agent in the subject, e.g., by imaging the subject (e.g., imaging at least a portion of the brain of the subject). For example, a NMR-detectable antibody to a TWEAK receptor can be used to identify Fn14 overexpressing cells at site of stroke damage.

Results of the evaluating can be used to provide a risk for stroke or an assessment of stroke status, e.g., by comparison to a reference, e.g., a reference value for a normal subject, a control subject, or a value determined, e.g., for a cohort of subjects.

The method can be used to evaluate a treatment for stroke. For example, the subject is receiving a treatment for stroke (e.g., a treatment using a TWEAK/TWEAK-R blocking agent, or other stroke treatment). The subject can be evaluated before, during, or after receiving the treatment, e.g., multiple times during the course of treatment.

The method can be used to identify a subject for stroke treatment. The subject can be identified as a subject suited for stroke treatment as a function of results of the evaluating, e.g., the results show similarity to, e.g., statistically significant similarity to, a reference value indicative of a subject requiring a stroke treatment. For example, elevated TWEAK or TWEAK receptor expression can be indicative of a subject who can be treated with a TWEAK/TWEAK-R blocking agent or other stroke treatment.

The method can also be used to select a patient population for treatment. Expression of TWEAK or a TWEAK receptor is evaluated for one or more subjects. A set of one or more subjects who have elevated expression of TWEAK or a TWEAK receptor relative to a reference are selected. The subjects of the set are administered an agent that blocks a TWEAK/TWEAK-R interaction or activity or other treatment for stroke.

In another aspect, this disclosure features a method that includes a) determining the identity of at least one nucleotide in the TWEAK and/or TWEAK receptor locus of a subject; and b) creating a record which includes information about the identity of the nucleotide and information relating to a stroke-related parameter of the subject, wherein the stroke-related parameter is other than the genotype of TWEAK or TWEAK receptor genes. The method can be used, e.g., for gathering genetic information. In one embodiment, the determining includes evaluating a sample including human genetic material from the subject. A related method includes: a) evaluating a parameter of a TWEAK and/or TWEAK receptor molecule (a TWEAK/TWEAK-R parameter) from a mammalian subject; and b) evaluating a stroke-related parameter of the subject wherein the stroke-related parameter is other than the parameter of (a).

The methods can also include c) recording information about the TWEAK/TWEAK-R parameter and information about the stroke-related parameter, wherein the information about the parameter and information about the phenotypic trait are associated with each other in a record, e.g., a database. For example, the stroke-related parameter is a phenotypic trait of the subject, e.g., a stroke-related parameter described herein.

In one embodiment, the TWEAK and/or TWEAK receptor molecule is a polypeptide and the TWEAK/TWEAK-R parameter includes information about a TWEAK/TWEAK-R polypeptide. In another embodiment, the TWEAK and/or TWEAK receptor molecule is a nucleic acid that encodes TWEAK or a TWEAK receptor and the TWEAK/TWEAK-R parameter includes information about identity of a nucleotide in the TWEAK/TWEAK-R gene. Other parameters can relate to TWEAK and/or TWEAK receptor expression, activity, modification, or localization (e.g., subcellular or organismal).

In an embodiment, the subject is an embryo, blastocyst, or fetus. In another embodiment, the subject is a post-natal human, e.g., a child or an adult (e.g., at least 20, 30, 40, 50, 60, or 70 years of age).

In one embodiment, step b) is performed before or concurrent with step a). In one embodiment, the human genetic material includes DNA and/or RNA.

The method can further include comparing the TWEAK/TWEAK-R parameter to reference information, e.g., information about a corresponding nucleotide from a reference sequence. In one embodiment, the reference subject did not exhibit stroke, e.g., at least prior to the time at which a nucleic acid from the reference subject and/or the reference subject's family history is not associated with stroke. In another embodiment, the reference sequence is from a reference subject that has stroke, e.g., a stroke at an age of less than age 60, 55, 50, or 45.

In one embodiment, the method further includes comparing the nucleotide to a corresponding nucleotide from a genetic relative or family member (e.g., a parent, grandparent, sibling, progeny, prospective spouse, etc.).

In one embodiment, the method further includes evaluating risk or determining diagnosis of stroke in the subject as a function of the genotype.

In one embodiment, the method further includes recording information about the TWEAK/TWEAK-R parameter and stroke-related parameter, e.g., in a database. For example, the information is recorded in linked fields of a database (e.g., TWEAK/TWEAK-R parameter is linked to at least one of: corresponding TWEAK/TWEAK-R parameter and/or data regarding comparison with the reference sequence). The nucleotide can be located in an exon, intron, or regulatory region of the TWEAK/TWEAK-R gene. For example, the nucleotide is a SNP. In one embodiment, a plurality of nucleotides (e.g., at least 10, 20, 50, 100, 500, or 1000 nucleotides (e.g., consecutive or non-consecutive)) in the TWEAK/TWEAK-R locus are evaluated. In another embodiment, a single nucleotide is evaluated.

In one embodiment, the method includes one or more of: evaluating a nucleotide position in the TWEAK/TWEAK-R locus on both chromosomes of the subject; recording the information (e.g., as phased or unphased information); aligning the genotyped nucleotides of the sample and the reference sequence; and identifying nucleotides that differ between the subject nucleotides and the reference sequence.

The method can be repeated for a plurality of subjects (e.g., at least 10, 25, 50, 100, 250, or 500 subjects).

In one embodiment, the method can include comparing the information of step a) and step b) to information in a database, and evaluating the association of the genotyped nucleotide(s) with stroke.

In another aspect, the invention features a computer readable record encoded with (a) a subject identifier, e.g., a patient identifier, (b) one or more results from an evaluation of the subject, e.g., a diagnostic evaluation described herein, e.g., the level of expression, level or activity of TWEAK or TWEAK receptor, in the subject, and optionally (c) a value for or related to stroke, e.g., a value correlated with disease status or risk with regard to stroke. In one embodiment, the invention features a computer medium having a plurality of digitally encoded data records. Each data record includes a value representing the expression, level, or activity of TWEAK or a TWEAK receptor, in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression or level of activity of genes other than TWEAK or TWEAK receptor (e.g., other genes associated with stroke, or other genes on an array). The data record can be structured as a table, e.g., a table that is part of a database such as a relational database (e.g., a SQL database of the Oracle or Sybase database environments). The invention also includes a method of communicating information about a subject, e.g., by transmitting information, e.g., transmitting a computer readable record described herein, e.g., over a computer network.

All cited patents, patent applications, and references are hereby incorporated by reference in their entireties. In the case of conflict, the present application controls.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows that TWEAK and Fn14 expression are induced by cerebral ischemia and OGD. A, Twenty-four hours after onset of cerebral ischemia, mRNA accumulation of TWEAK and Fn14 in the ischemic or contralateral cortex was determined by RT-real-time PCR. Values are means±SE (n= 12) expressed as percentage of the right hemisphere. *p< 0.02; ⁺p< 0.0003 (t test). B, In situ hybridization demonstrated upregulation of Fn14 mRNA 24 hours after onset of MCAO in the cortex adjacent to the infarct. There was no staining in the contralateral cortex. Similar results were obtained after 48 hours of MCAO. Scale bar, 200 μm. C, Immunocytochemistry with an Fn14-specific polyclonal serum or preimmune serum and an anti-Fn14 mAb or control (DMEM culture medium) revealed expression of Fn14 in cortical neurons. D, mRNA accumulation of TWEAK (left) and Fn14 (right) in cortical neurons was stimulated by OGD. After OGD for the times indicated, cells were incubated under normal conditions for 24 hours. Values are means±SE (n=6) measured in duplicate and expressed as percentage of control cells. ⁺p<0.006; *p<0.0001 (t test).

FIG. 2 shows that TWEAK induces cell death in primary cortical neurons of the mouse. A, Cortical neurons at 10 days in vitro were exposed to OGD for 4.5 hours and then incubated under standard conditions for 24 hours and stained by the TUNEL reaction. Neuronal cell death was reduced by the monoclonal hamster anti-TWEAK antibody AB.G11 (10 μg/ml). Values are means±SE of six experiments, each counted in quintuplicate, and are expressed relative to the control group treated with control hamster antibody. *p<0.0001 (Mann-Whitney U test). B, After exposure of neurons to rhTWEAK (100 ng/ml) or Fc-hTWEAK (100 ng/ml) for 24 hours, cells were stained by DAPI and by the TUNEL reaction. Both forms of TWEAK increased the number of cells with condensed nuclei that were TUNEL positive. C, Quantification of cells with condensed nuclei after DAPI staining (gray columns) or of TUNEL-positive cells (black columns). Values are means±SE of three experiments, each counted in quintuplicate, and are expressed as percentage of total cell number. C A, Camptothecin (10 μM). *p< 0.005 (ANOVA; LSD post hoc) applying to both methods of apoptosis detection. D, Quantification of histone-associated DNA fragments in cytosolic extracts of cortical neurons after rhTWEAK (100 ng/ml) and Fc-hTWEAK (100 ng/ml) treatment for 24 hr. Values are means±SE (n=4) and expressed relative to the untreated control group. *p< 0.03 (ANOVA; LSD post hoc).

FIG. 3 shows that TWEAK activates NF-κB through Fn14 and IKK in cortical neurons. Cortical neurons were transfected with pNF-κB-Luc, a luciferase fusion gene that contains five binding sites for NF-κB, and were stimulated by TWEAK for 24 hours. A, B, Fc-hTWEAK (A) and rhTWEAK (B) stimulated NF-κB activity in a concentration-dependent manner. C, D, The stimulation of NF-κB by Fc-hTWEAK (C) or rhTWEAK (D) in a concentration of 10 and 100 ng/ml was abrogated by the neutralizing anti-TWEAK antibody AB.G11 (10 μg/ml added 30 minutes before stimulation); however, stimulation by TNF-α (10 ng/ml) was not affected by AB.G11. Controls received an unspecific hamster Ig. *p<0.05; **p<0.0001 (ANOVA; LSD post hoc test). E, ITEM-4 (1 ng/ml), an anti-Fn14 mAb that blocks TWEAK-Fn14 interaction, was added to the medium 20 minutes before TWEAK. It partially blocked NF-κB stimulation by rhTWEAK (100 ng/ml). Controls received unspecific mouse Ig (1 μg/ml). ⁺p< 0.005 (ANOVA; LSD post hoc test). F, NF-κB stimulation by rhTWEAK or Fc-hTWEAK was inhibited by the IKK inhibitor BMS-345541 (25 μM). *p<0.04; **p<0.0001 (ANOVA; LSD post hoc test). Values are means±SE (n=9) of the luciferase activity expressed in percentage of untreated controls.

FIG. 4 shows that TWEAK-induced neuronal cell death is mediated by NF-κB. A, Cortical neurons were stimulated by drugs for 24 hours and then stained by the TUNEL reaction. The NF-κB inhibitor SN50 (10 μg/ml) increased the basal rate of TUNEL-positive cells but reduced the proapoptotic effect of rhTWEAK (100 ng/ml) and Fc-hTWEAK (100 ng/ml) significantly. Values are means±SE (n=3), each performed in duplicate, and are expressed relative to the untreated control. *p<0.0001 (ANOVA; LSD post hoc test). B, Cortical neurons from mice expressing the NF-κB super-repressor (IBSR) or from wild-type littermates were transfected with the luciferase fusion gene pNF-κB-Luc and the renilla luciferase control plasmid phRL-TK. rhTWEAK (100 ng/ml) stimulated NF-κB less in neurons from IκBα-SR mice than in neurons from wild-type littermates. Values are means±SE of luciferase activity expressed as percentage of the un-stimulated control of the same genotype (n=18-22). ⁺p<0.001 (ANOVA; post hoc test). C, IκBα-SR-expressing neurons were protected from cell death induced by rhTWEAK (100 ng/ml). After 24 hours of treatment, apoptotic cells were determined by the TUNEL reaction. Values are means±SE of four experiments, each counted in quintuplicate, and are expressed relative to the untreated wild-type group, *p<0.0001 (Mann-Whitney U test).

FIG. 5 shows that inhibition of TWEAK reduced the infarct size. Mice were injected with the neutralizing monoclonal anti-TWEAK antibody AB.G11 or an unspecific monoclonal hamster Ig (HA; 200 μg) intraperitoneally immediately before onset of cerebral ischemia and were killed 48 hours later. The infarcts were visualized by silver staining. Typical coronal sections are shown at the top. Below, means and individual values of the corrected infarct volume are shown (n=12-13). *p< 0.05 (t test).

FIG. 6 shows that anti-TWEAK antibody (AB.G11) treatment reduces astrocyte activation after MCAO. Mice were treated with anti-TWEAK antibody AB.G11 or an unspecific control antibody (HA4/8). Coronal brain slices from each mouse were prepared and stained with anti-GFAP antibody. Two regions of the brain (R1 and R2) from each mouse were stained. The quantification of GFAP positive staining in ischemic vs. contralateral hemisphere showed significant reduction in the amount of astrocyte activation in ischemic side in region R1 of AB.G11 treated animals (p<0.01).

### DETAILED DESCRIPTION

The results presented herein demonstrate, among other things, that administration of a TWEAK/TWEAK-R blocking agent, e.g., a TWEAK antibody, can reduce infarct size in vivo in a model of cerebral ischemia. Accordingly, a TWEAK/TWEAK-R blocking agent can be administered to heat stroke, e.g., alone or in combination with another TWEAK/TWEAK-R blocking agent or another treatment for stroke.

The term “treating” refers to administering a therapy in an amount, manner, and/or mode effective to improve or prevent a condition, symptom, or parameter associated with a disorder (e.g., stroke or other disorder described herein) or to prevent onset, progression, or exacerbation of the disorder (including secondary damage caused by the disorder, e.g., stroke), to either a statistically significant degree or to a degree detectable to one skilled in the art. Accordingly, heating can achieve therapeutic and/or prophylactic benefits. An effective amount, manner, or mode can vary depending on the subject and may be tailored to the subject.

### TWEAK/TWEAK Receptor Blocking Agents

A variety of agents can be used as a TWEAK/TWEAK-R blocking agent to treat stroke. The agent may be any type of compound (e.g., small organic or inorganic molecule, nucleic acid, protein, or peptide mimetic) that can be administered to a subject. In one embodiment, the blocking agent is a biologic, e.g., a protein having a molecular weight of between 5-300 kDa. For example, a TWEAK/TWEAK-R blocking agent may inhibit binding of TWEAK to a TWEAK receptor or may prevent TWEAK-mediated NF-κB activation. A typical TWEAK/TWEAK-R blocking agent can bind to TWEAK or a TWEAK receptor, e.g., Fn14. A TWEAK/TWEAK-R blocking agent that binds to TWEAK may alter the conformation of TWEAK or a TWEAK receptor, block the binding site on TWEAK or a TWEAK receptor, or otherwise decrease the affinity of TWEAK for a TWEAK receptor or prevent the interaction between TWEAK and a TWEAK receptor. A TWEAK/TWEAK-R blocking agent (e.g., an antibody) may bind to TWEAK or to a TWEAK receptor with a K_{d} of less than 10⁻⁶, 10⁻⁷, 10⁻⁸, 10⁻⁹, or 10⁻¹⁰ M. In one embodiment, the blocking agent binds to TWEAK with an affinity at least 5, 10, 20, 50, 100, 200, 500, or 1000-fold better than its affinity for TNF or another TNF superfamily member (other than TWEAK). In one embodiment, the blocking agent binds to the TWEAK receptor with an affinity at least 5, 10, 20, 50, 100, 200, 500, or 1000-fold better than its affinity for the TNF receptor or a receptor for another TNF superfamily member. A preferred TWEAK/TWEAK-R blocking agent specifically binds TWEAK or a TWEAK receptor, such as a TWEAK or TWEAK receptor specific antibody.

Exemplary TWEAK protein molecules include human TWEAK (e.g., AAC51923; shown as SEQ ID NO:1), mouse TWEAK (e.g., NP_{—}035744.1), rat TWEAK (e.g., XP_{—}340827.1), and Pan troglodytes TWEAK (e.g., XP_{—}511964.1). Also included are proteins that include an amino acid sequence at least 90, 92, 95, 97, 98, 99% identical and completely identical to the mature processed region of the aforementioned TWEAK proteins (e.g., an amino acid sequence at least 90, 92, 95, 97, 98, 99% identical or completely identical to amino acids X₁-249 of SEQ ID NO:1, where amino acid X₁ is selected from the group of residues 75-115 of SEQ ID NO:1, e.g., X₁ is residue Arg 93 of SEQ ID NO:1) and proteins encoded by a nucleic acid that hybridizes under high stringency conditions to a human, mouse, rat, or Pan troglodytes gene encoding a naturally occurring TWEAK protein. Preferably, a TWEAK protein, in its processed mature form, is capable of providing at least one TWEAK activity, e.g., ability to activate Fn14 and/or cell death in cortical neurons.

Exemplary TWEAK receptor molecules include Fn14. Exemplary Fn14 protein molecules include human Fn14 (e.g., NP_{—}057723.1; shown as SEQ ID NO:2), mouse Fn14 (e.g., NP_{—}038777.1), and rat Fn14 (e.g., NP_{—}851600.1), as well as soluble proteins that include an amino acid sequence at least 90, 92, 95, 97, 98, 99% identical or 100% identical to the extracellular domain of Fn14 (and TWEAK-binding fragments thereof) and proteins encoded by a nucleic acid that hybridizes under high stringency conditions to a human, mouse, rat, or Pan troglodytes gene encoding a naturally-occurring Fn14 protein. Preferably, an Fn14 protein useful in the methods described herein is a soluble Fn14 (lacking a transmembrane domain) that includes a region that binds to a TWEAK protein, e.g., an amino acid sequence at least 90, 92, 95, 97, 98, or 99% identical, or completely identical, to amino acids 28-X₁ of SEQ ID NO:2, where amino acid X₁ is selected from the group of residues 68 to 80 of SEQ ID NO:2.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. High stringency hybridization conditions include hybridization in 6×SSC at about 45° C., followed by one or more washes in 0.2×SSC, 0.1% SDS at 65° C., or substantially similar conditions.

Exemplary TWEAK/TWEAK-R blocking agents include antibodies that bind to TWEAK or a TWEAK receptor and soluble forms of the TWEAK receptor that compete with cell surface TWEAK receptor for binding to TWEAK. An example of a soluble form of the TWEAK receptor is an Fc fusion protein that includes at least a portion of the extracellular domain of a TWEAK receptor (e.g., a soluble TWEAK-binding fragment of a TWEAK receptor, e.g., Fn14), referred to as TWEAK-R-Fc. Other soluble forms of TWEAK receptor, e.g., forms that do not include an Fc domain, can also be used. Antibody blocking agents are further discussed below. Other types of blocking agents, e.g., small molecules, nucleic acid or nucleic acid-based aptamers, and peptides, can be isolated by screening, e.g., as described in Jhaveri et al. (2000) Nat. Biotechnol, 18:1293 and U.S. Pat. No. 5,223,409. Exemplary assays for determining if an agent binds to TWEAK or a TWEAK receptor and for determining if an agent modulates a TWEAK/TWEAK-R interaction are described, e.g., in U.S. Pub. App. No. 2004-0033225.

An exemplary soluble form of the TWEAK-R protein includes a region of the TWEAK-R protein that binds to TWEAK, e.g., about amino acids 32-75, 31-75, 31-78, or 28-79 of SEQ ID NO:2. This region can be physically associated, e.g., fused to another amino acid sequence, e.g., an Fc domain, at its N- or C-terminus. The region from TWEAK receptor can be spaced by a linker from the heterologous amino acid sequence. U.S. Pat. No. 6,824,773 describes an exemplary TWEAK receptor fusion protein.

### Antibodies

Exemplary TWEAK/TWEAK-R blocking agents include antibodies that bind to TWEAK and/or a TWEAK receptor. In one embodiment, the antibody inhibits the interaction between TWEAK and a TWEAK receptor, e.g., by physically blocking the interaction, decreasing the affinity of TWEAK and/or a TWEAK receptor for its counterpart, disrupting or destabilizing TWEAK complexes, sequestering TWEAK or a TWEAK receptor, or targeting TWEAK or a TWEAK receptor for degradation. In one embodiment, the antibody can bind to TWEAK or a TWEAK receptor at one or more amino acid residues that participate in the TWEAK/TWEAK-R binding interface. Such amino acid residues can be identified, e.g., by alanine scanning. In another embodiment, the antibody can bind to residues that do not participate in the TWEAK/TWEAK-R binding. For example, the antibody can alter a conformation of TWEAK or a TWEAK receptor and thereby reduce binding affinity, or the antibody may sterically hinder TWEAK/TWEAK-R binding. In one embodiment, the antibody can prevent activation of a TWEAK/TWEAK-R mediated event or activity (e.g., NF-κB activation).

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A. et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev. Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally-occurring variable domain. For example, the sequence may omit one, two, or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with TWEAK or a TWEAK receptor.

The VH or VL chain of the antibody can further include all or part of a heavy or light chain constant region (respectively), to thereby form a heavy immunoglobulin chain (HC) or light immunoglobulin chain (LC), respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins, or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

### Antibody Generation

Antibodies that bind to TWEAK or a TWEAK receptor can be generated by a variety of means, including immunization, e.g., using an animal, or in vitro methods such as phage display. All or part of TWEAK or a TWEAK receptor can be used as an immunogen or as a target for selection. For example, TWEAK or a fragment thereof, or a TWEAK receptor or a fragment thereof, can be used as an immunogen. In one embodiment, the immunized animal contains immunoglobulin-producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nat. Gen. 7:13-21; U.S. Pub. App. No. 2003-0070185; U.S. Pat. No. 5,789,650; and PCT Pub. No. WO 96/34096.

Non-human antibodies to TWEAK or a TWEAK receptor can also be produced, e.g., in a rodent. The non-human antibody can be humanized, e.g., as described in EP 239 400; U.S. Pat. Nos. 6,602,503; 5,693,761; and 6,407,213, deimmunized, or otherwise modified to make it effectively human.

EP 239 400 (Winter et al.) describes altering antibodies by substitution (within a given variable region) of their complementarity determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody. Other methods for humanizing antibodies can also be used. For example, other methods can account for the three-dimensional structure of the antibody, framework positions that are in three-dimensional proximity to binding determinants, and immunogenic peptide sequences. See, e.g., PCT Pub. No. WO 90/07861; U.S. Pat. Nos. 5,693,762; 5,693,761; 5,585,089; and 5,530,101; Tempest et al. (1991) Biotechnology 9:266-271 and U.S. Pat. No. 6,407,213.

Fully human monoclonal antibodies that bind to TWEAK or a TWEAK receptor can be produced, e.g., using in vitro-primed human splenocytes, as described by Boerner et al. (1991) J. Immunol. 147:86-95. They may be prepared by repertoire cloning as described by Persson et al. (1991) Proc. Natl. Acad. Sci. USA 88:2432-2436 or by Huang and Stollar (1991) J. Immunol. Methods 141:227-236; also U.S. Pat. No. 5,798,230. Large nonimmunized human phage display libraries may also be used to isolate high affinity antibodies that can be developed as human therapeutics using standard phage technology (see, e.g., Hoogenboom et al. (1998) Immunotechnology 4:1-20; Hoogenboom et al. (2000) Immunol. Today 2:371-8; and U.S. Pub. App. No. 2003-0232333).

### Antibody and Protein Production

Antibodies and other proteins described herein can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-135), Hanseula, or Saccharomyces.

Antibodies, particularly full length antibodies, e.g., IgG's, can be produced in mammalian cells. Exemplary mammalian host cells for recombinant expression include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017). Exemplary selectable marker genes include the dihydrofolate reductase (DHFR) gene (for use in dhfr host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus, and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies (and Fc fusions) may also include modifications, e.g., modifications that alter Fc function, e.g., to decrease or remove interaction with an Fc receptor or with Clq, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some proteins that include an Fc domain, the antibody/protein production system may be designed to synthesize antibodies or other proteins in which the Fc region is glycosylated. For example, the Fc domain of IgG molecules is glycosylated at asparagine 297 in the CH2 domain. The Fc domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the protein of interest, e.g., an antibody or Fc fusion protein. The protein can be purified from the milk, or for some applications, used directly.

Methods described in the context of antibodies can be adapted to other proteins, e.g., Fc fusions and soluble receptor fragments.

### Nucleic Acid Blocking Agents

In certain implementations, nucleic acid blocking agents are used to decrease expression of an endogenous gene encoding TWEAK or a TWEAK receptor, e.g., Fn14. In one embodiment, the nucleic acid antagonist is an siRNA that targets mRNA encoding TWEAK or a TWEAK receptor. Other types of blocking nucleic acids can also be used, e.g., a dsRNA, a ribozyme, a triple-helix former, or an antisense nucleic acid.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature 411:494-498; Yang et al. (2002) Proc. Natl. Acad. Sci. USA 99:9942-9947, U.S. Pub. App. Nos. 2003-0166282, 2003-0143204, 2004-0038278, and 2003-0224432.

Anti-sense agents can include, for example, from about 8 to about 80 nucleobases (i.e., from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA (e.g., an mRNA encoding TWEAK or a TWEAK receptor) can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding TWEAK or a TWEAK receptor. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴-(C₁-C₁₂) alkylaminocytosines and N⁴,N⁴-(C₁-C₁₂) dialkylaminocytosines. Modified nucleobases may also include 7-substituted-5-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶-(C₁-C₁₂) alkylaminopurines and N⁶,N⁶-(C₁-C₁₂) dialkylaminopurines, including N⁶-methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. Nos. 4,987,071; 5,116,742; and 5,093,246; Woolf et al. (1992) Proc. Natl. Acad. Sci. USA 89:7305-7309; Antisense RNA and DNA, D. A. Melton, Ed., Cold Spring Harbor Laboratory, Cold Spring Harbor, N.Y. (1988); Haseloff and Gerlach (1988) Nature 334:585-591; Helene, C. (1991) Anticancer Drug Des. 6:569-584; Helene (1992) Ann. N.Y. Acad. Sci. 660:27-36; and Maher (1992) Bioassays 14:807-815.

The nucleic acids described herein, e.g., an anti-sense nucleic acid described herein, can be incorporated into a gene construct to be used as a part of a gene therapy protocol to deliver nucleic acids that can be used to express and produce agents, e.g., anti-sense nucleic acids, within cells. Expression constructs of such components may be administered in any biologically-effective carrier, e.g., any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g., antibody conjugated) polylysine conjugates, gramacidin S, artificial viral envelopes or other such intracellular earners, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo particularly into humans. These vectors provide efficient delivery of genes into cells, and the transferred nucleic acids are stably integrated into the chromosomal DNA of the host. Protocols for producing recombinant retroviruses and for infecting cells in vitro or in vivo with such viruses can be found in Current Protocols in Molecular Biology, Ausubel, F. M. et al. (eds.) Greene Publishing Associates (1989), Sections 9.10-9.14 and other standard laboratory manuals. Examples of suitable retroviruses include pLJ, pZIP, pWE, and pEM which are known to those skilled in the art. Examples of suitable packaging virus lines for preparing both ecotropic and amphotropic retroviral systems include *Crip, *Cre, *2, and *Am. Retroviruses have been used to introduce a variety of genes into many different cell types, including epithelial cells, in vitro and/or in vivo (see, for example, Eglitis et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116 and 4,980,286; PCT Pub. Nos. WO 89/07136, WO 89/02468, WO 89/05345, and WO 92/07573).

Another viral gene delivery system utilizes adenovirus-derived vectors. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are known to those skilled in the art.

Yet another viral vector system useful for delivery of the subject gene is the adeno-associated virus (AAV). See, for example, Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973.

### Artificial Transcription Factors

Artificial transcription factors can also be used to regulate expression of TWEAK and/or a TWEAK receptor. The artificial transcription factor can be designed or selected from a library, e.g., for ability to bind to a sequence in an endogenous gene encoding TWEAK or a TWEAK receptor, e.g., in a regulatory region, e.g., the promoter. For example, the artificial transcription factor can be prepared by selection in vitro (e.g., using phage display, U.S. Pat. No. 6,534,261) or in vivo, or by design based on a recognition code (see, e.g., PCT Pub. No. WO 00/42219 and U.S. Pat. No. 6,511,808). See, e.g., Rebar et al. (1996) Methods Enzymol. 267:129; Greisman and Pabo (1997) Science 275:657; Isalan et al. (2001) Nat. Biotechnol. 19:656; and Wu et al. (1995) Proc. Natl. Acad. Sci. USA 92:344 for, among other things, methods for creating libraries of varied zinc finger domains.

Optionally, an artificial transcription factor can be fused to a transcriptional regulatory domain, e.g., an activation domain to activate transcription or a repression domain to repress transcription. In particular, repression domains can be used to decrease expression of endogenous genes encoding TWEAK or a TWEAK receptor. The artificial transcription factor can itself be encoded by a heterologous nucleic acid that is delivered to a cell or the protein itself can be delivered to a cell (see, e.g., U.S. Pat. No. 6,534,261). The heterologous nucleic acid that includes a sequence encoding the artificial transcription factor can be operably linked to an inducible promoter, e.g., to enable fine control of the level of the artificial transcription factor in the cell, e.g., a neuronal or glial cell, e.g., at or near a site of stroke injury.

### Stroke

Stroke is a general term for acute brain damage resulting from disease of blood vessels. Stroke can be classified into at least two main categories: hemorrhagic stroke (resulting from leakage of blood outside of the normal blood vessels) and ischemic stroke (cerebral ischemia due to lack of blood supply). Some events that can cause ischemic stroke include thrombosis, embolism, and systemic hypoperfusion (with resultant ischemia and hypoxia).

Stroke generally causes neuronal death and injury in the brain by oxygen deprivation and secondary events. The area of the brain that dies as a result of the lack of blood supply or other damage is called an infarct. In some cases, the treatments described herein can be used to reduce or minimize the size of an infarct, e.g., by reducing secondary events that cause neuronal death or injury.

Obstruction of a cerebral artery resulting from a thrombus which has built up on the wall of a brain artery is generally called cerebral thrombosis. In cerebral embolism, the occlusive material blocking the cerebral artery arises downstream in the circulation (e.g., an embolus is carried to the cerebral artery from the heart). Because it is difficult to discern whether a stroke is caused by thrombosis or embolism, the term thromboembolism is used to cover both these types of stroke. Systemic hypoperfusion may arise as a consequence of decreased blood levels, reduced hematocrit, low blood pressure, or inability of the heart to pump blood adequately.

Thrombolytic agents, such as tissue plasminogen activator (t-PA), have been used in the treatment of thromboembolic stroke. These molecules function by lysing the thrombus causing the ischemia. Such drugs are believed to be most useful if administered as soon as possible after acute stroke (preferably within 3 hours) in order to at least partially restore cerebral blood flow in the ischemic region and to sustain neuronal viability. A TWEAK/TWEAK-R blocking agent can be used, instead of or in combination with, such thrombolytic agents, to achieve a therapeutic benefit in a subject who has experienced a thromboembolic stroke.

Because thrombolytic agents exacerbate bleeding, their use in hemorrhagic stroke is contra-indicated. However, a TWEAK/TWEAK-R blocking agent can be used to provide therapeutic benefit in cases of hemorrhagic stroke.

Further, a TWEAK/TWEAK-R blocking agent can be administered as a prophylactic stroke therapy, or as a component thereof, e.g., to a subject who has experienced a TIA or is exhibiting symptoms of TIA. When symptoms of stroke last less than 24 hours and the subject recovers completely, the subject is said to have undergone a transient ischemic attack (TIA). The symptoms of TIA include a temporary impairment of speech, vision, sensation, or movement. Because a TIA is often thought to be a prelude to full-scale stroke, subjects having suffered a TIA are candidates for prophylactic stroke therapy, e.g., with a TWEAK/TWEAK-R blocking agent alone or in combination with another agent, e.g., an anticoagulation agent (e.g., coumarin and heparin) or an antiplatelet agent (such as aspirin and ticlopidine).

### Other Stroke Treatments

A stroke treatment can involve the use of one or more TWEAK/TWEAK-R blocking agent that can be used in combination with one or more stroke treatments. The term “in combination” refers to both administration of the TWEAK/TWEAK-R blocking agent and the other treatment to the subject such that both treatments provide a concurrent benefit. The treatments can be administered at the same time, but also at separate times, e.g., at separate times that are within a specified interval, e.g., within the same 48, 24, 12, 6, 2, or 1 hour.

Treatments that can be administered in combination with a TWEAK/TWEAK-R blocking agent include: a thrombolytic agent (e.g., streptokinase, acylated plasminogen-streptokinase activator complex (APSAC), urokinase, single-chain urokinase-plasminogen activator (scu-PA), anti-inflammatory agents, thrombin-like enzymes from snake venoms such as ancrod, tissue plasminogen activator (t-PA), and biologically active variants of each of the above); an anticoagulant (e.g., warfarin); an antiplatelet drug (e.g., aspirin); an anti-CD18 antibody; an anti-CD11a antibody; an anti-ICAM-1 antibody; an anti-VLA-4 antibody; a carotid endarterectomy; angioplasty; insertion of a stent; and an alternative medicine (e.g., acupuncture, traditional Chinese medicine, meditation, massage, hyperbaric oxygen treatment, or conductive pedagogy).

Particular examples of combination treatments include administering a TWEAK/TWEAK-R blocking agent to a subject who has experienced a stroke shortly after the onset of stroke symptoms and at the same time as another treatment, such as t-PA. The following day, the subject can further commence daily treatments with an anti-platelet drug to prevent a future stroke and later receive additional doses of the TWEAK/TWEAK-R blocking agent, to maintain bioavailability of the blocking agent. As another example, a subject who has experienced a TIA may begin TWEAK/TWEAK-R blocking agent treatment immediately after diagnosis of the TIA at a dose that provides a biological effect for at least a week, and then begin anti-platelet therapy the following day.

### Stroke Risk Factors

Risk factors for stroke can be used to identify a subject who can be provided with a prophylatic dose of a TWEAK/TWEAK-R blocking agent or who should be monitored for further signs that treatment with a TWEAK/TWEAK-R blocking agent is required. In some cases, the subject is treated if the subject has two, three, or four of more of risk factors, e.g., factors listed below.

High blood pressure: High blood pressure (140/90 mm Hg or higher) is a highly significant risk factor for stroke.

Tobacco use: Cigarette smoking is a major, preventable risk factor for stroke. The nicotine and carbon monoxide in tobacco smoke reduce the amount of oxygen in the blood. They also damage the walls of blood vessels, making clots more likely to form. Using some kinds of birth control pills combined with smoking cigarettes greatly increases stroke risk.

Diabetes mellitus: Diabetes is defined as a fasting plasma glucose (blood sugar) of 126 mg/dL or more measured on two occasions. While diabetes is treatable, having it still increases a person's risk of stroke. Many people with diabetes also have high blood pressure, high blood cholesterol, and are overweight. These additional factors further increase risk of stroke.

Carotid or other artery disease: The carotid arteries in the neck supply blood to the brain. A carotid artery narrowed by fatty deposits from atherosclerosis (plaque buildups in artery walls) may become blocked by a blood clot. Carotid artery disease is also called carotid artery stenosis.

Peripheral artery disease: Subjects with peripheral artery disease have a higher risk of carotid artery disease, which raises their risk of stroke. Peripheral artery disease is the narrowing of blood vessels carrying blood to leg and arm muscles. It is caused by fatty buildups of plaque in artery walls.

Atrial fibrillation raises the risk for stroke. The upper chambers of the heart quiver instead of beating effectively, which can let the blood pool and clot. If a clot breaks off, enters the bloodstream and lodges in an artery leading to the brain, a stroke results.

Other heart disease: Subjects with coronary heart disease or heart failure have a higher risk of stroke than those with hearts that work normally. Dilated cardiomyopathy (an enlarged heart), heart valve disease, and some types of congenital heart defects also raise the risk of stroke.

Transient ischemic attacks (TIAs): TIAs are “warning strokes” that produce stroke-like symptoms but no lasting damage. Recognizing and treating TIAs can reduce the risk of a major stroke.

Certain blood disorders: A high red blood cell count thickens the blood and makes clots more likely. This raises the risk of stroke. Sickle cell disease (also called sickle cell anemia) is a genetic disorder that mainly affects African Americans. “Sickled” red blood cells are less able to carry oxygen to the body's tissues and organs and tend to stick to blood vessel walls, which can block arteries to the brain and cause a stroke.

High blood cholesterol: A high level of total cholesterol in the blood (240 mg/dL or higher) is a major risk factor for heart disease, which raises the risk of stroke. High levels of LDL cholesterol (greater than 100 mg/dL) and triglycerides (blood fats, 150 mg/dL or higher) increase the risk of stroke in people with previous coronary heart disease, ischemic stroke or transient ischemic attack (TIA). Low levels (less than 40 mg/dL) of HDL cholesterol also may raise stroke risk.

Physical inactivity and obesity: Being inactive, obese, or both can increase the risk of high blood pressure, high blood cholesterol, diabetes, heart disease, and stroke.

Excessive substance abuse: Drinking excessive amounts of alcohol and intravenous drug use can also increase risk for stroke.

Increasing age: Although subjects of all ages, including children, have strokes, the older the subject is, the greater the risk for stroke. For example, risk can be much greater over the age of 55, 60, 70, 80, or 85.

Sex (gender): Stroke is more common in men than in women. In most age groups, more men than women will have a stroke in a given year. However, women account for more than half of all stroke deaths. Women who are pregnant have a higher stroke risk.

Heredity (family history): The stroke risk is greater if a parent, grandparent, sister, or brother has had a stroke. Similarly, certain ethnic backgrounds can lead to an increased risk for stroke.

Prior stroke or heart attack: A subject who has had a stroke or a heart attack is at much higher risk of subsequently having a stroke.

### Stroke Assessment Criteria

The ability of a TWEAK/TWEAK-R blocking agent to treat a subject having or at risk for stroke can be evaluated, subjectively or objectively, e.g., using a variety of criteria. A number of assessment tools are available to provide the evaluation.

Exemplary prehospital stroke assessment tools include the Cincinnati Stroke Scale and the Los Angeles Prehospital Stroke Screen (LAPSS). Acute assessment scales include, e.g., the Canadian Neurological Scale (CNS), the Glasgow Coma Scale (GCS), the Hempispheric Stroke Scale, the Hunt & Hess Scale, the Mathew Stroke Scale, the Mini-Mental State Examination (MMSE), the NIH Stroke Scale (NIHSS), the Orgogozo Stroke Scale, the Oxfordshire Community Stroke Project Classification (Bamford), and the Scandinavian Stroke Scale. Functional assessment scales include the Berg Balance Scale, the Modified Rankin Scale, the Stroke Impact Scale (SIS), and the Stroke Specific Quality of Life Measure (SS-QOL). Outcome assessment tools include the American Heart Association Stroke Outcome Classification (AHA SOC), the Barthel Index, the Functional Independence Measurement (FIM™), the Glasgow Outcome Scale (GOS), and the Health Survey SF-36™ & SF-12™. Other diagnostic and screening tests include the Action Research Arm Test, the Blessed-Dementia Scale, the Blessed-Dementia Information-Memory-Concentration Test, the DSM-IV criteria for the diagnosis of vascular dementia, the Hachinkski Ischaemia Score, the Hamilton Rating Scale for Depression, the NINDS-AIREN criteria for the diagnosis of vascular dementia, the Orpington Prognostic Score, and the Short Orientation-Memory-Concentration Test.

An evaluation can be performed before and/or after the administration of a TWEAK or TWEAK receptor blocking agent.

### Pharmaceutical Compositions

A TWEAK/TWEAK-R blocking agent (e.g., an antibody or soluble TWEAK-R protein, e.g., TWEAK-R-Fc) can be formulated as a pharmaceutical composition, e.g., for administration to a subject to treat stroke. Typically, a pharmaceutical composition includes a pharmaceutically acceptable carrier. As used herein, “pharmaceutically acceptable earner” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see, e.g., Berge, S. M. et al. (1977) J. Pharm. Sci. 66:1-19).

The TWEAK/TWEAK-R blocking agent can be formulated according to standard methods. Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

In one embodiment, the TWEAK/TWEAK-R blocking agent (e.g., an antibody or TWEAK-R-Fc) can be formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

The pharmaceutical compositions may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants. Prolonged absorption of injectable compositions can be brought about by including in the composition an agent that delays absorption, for example, monostearate salts and gelatin.

In certain embodiments, the TWEAK/TWEAK-R blocking agent may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

A TWEAK/TWEAK-R blocking agent (e.g., an antibody or soluble TWEAK-R protein) can be modified, e.g., with a moiety that improves its stabilization and/or retention in circulation, e.g., in blood, serum, or other tissues, e.g., by at least 1.5, 2, 5, 10, or 50-fold. The modified blocking agent can be evaluated to assess whether it can reach sites of damage after a stroke (e.g., by using a labeled form of the blocking agent).

For example, the TWEAK/TWEAK-R blocking agent (e.g., an antibody or soluble TWEAK-R protein) can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used.

For example, a TWEAK or a TWEAK receptor binding antibody can be conjugated to a water-soluble polymer, e.g., a hydrophilic polyvinyl polymer, e.g., polyvinylalcohol or polyvinylpyrrolidone. A non-limiting list of such polymers includes polyalkylene oxide homopolymers such as polyethylene glycol (PEG) or polypropylene glycols, polyoxyethylenated polyols, copolymers thereof, and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; and branched or unbranched polysaccharides.

When the TWEAK/TWEAK-R blocking agent (e.g., an antibody or soluble TWEAK-R protein) is used in combination with a second agent, the two agents can be formulated separately or together. For example, the respective pharmaceutical compositions can be mixed, e.g., just prior to administration, and administered together or can be administered separately, e.g., at the same or different times.

### Administration

The TWEAK/TWEAK-R blocking agent (e.g., an antibody or soluble TWEAK-R protein) can be administered to a subject, e.g., a human subject, by a variety of methods. For many applications, the route of administration is one of: intravenous injection or infusion (IV), subcutaneous injection (SC), intraperitoneally (IP), or intramuscular injection. In some cases, administration may be directly into the CNS, e.g., intrathecal or intracerebroventricular (ICV). The blocking agent can be administered as a fixed dose, or in a mg/kg dose.

The dose can also be chosen to reduce or avoid production of antibodies against the TWEAK/TWEAK-R blocking agent.

The route and/or mode of administration of the blocking agent can also be tailored for the individual case, e.g., by monitoring the subject, e.g., using tomographic imaging, neurological exam, and standard parameters associated with stroke, e.g., the stroke assessment criteria discussed above.

Dosage regimens are adjusted to provide the desired response, e.g., a therapeutic response or a combinatorial therapeutic effect. Generally, any combination of doses (either separate or co-formulated) of the TWEAK/TWEAK-R blocking agent (e.g., an antibody) (and optionally a second agent) can be used in order to provide a subject with the agent in bioavailable quantities. For example, doses in the range of 1 mg/kg-100 mg/kg, 0.5-20 mg/kg, or 1-10 mg/kg can be administered.

Dosage unit form or “fixed dose” as used herein refers to physically discrete units suited as unitary dosages for the subjects to be heated; each unit contains a predetermined quantity of active compound calculated to produce the desired therapeutic effect in association with the required pharmaceutical carrier and optionally in association with the other agent, if used.

The TWEAK/TWEAK-R blocking agent may be administered at least once between about 10 minutes to about 48 hours, more preferably between about 10 minutes and 24 hours, more preferably within 3 hours, after the onset of stroke symptoms or manifestation. Single or multiple dosages may be given. Alternatively, or in addition, the blocking agent may be administered via continuous infusion. The treatment can continue for days, weeks, months, or even years so as to minimize ischemic damage from the stroke, to minimize damage from post-stroke inflammatory events, and/or to prevent another stroke or to minimize damage that might result from a subsequent stroke.

If a subject is at risk for stroke or has suffered a TIA, the blocking agent can be administered before the onset of a stroke as a preventative measure. The duration of such preventative treatment can be a single dosage of the blocking agent or the treatment may continue (e.g., multiple dosages), for example, a subject at risk for stroke may be treated with the blocking agent for days, weeks, months, or even years so as to prevent a stroke from occurring.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter, e.g., a stroke parameter, or amelioration of at least one symptom of the disorder, e.g., stroke. A therapeutically effective amount is also one in which any toxic or detrimental effects of the composition are outweighed by the therapeutically beneficial effects.

### Devices and Kits

Pharmaceutical compositions that include the TWEAK/TWEAK-R blocking agent (e.g., an antibody or soluble TWEAK receptor) can be administered with a medical device. The device can designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include a TWEAK/TWEAK-R blocking agent, and can be configured to deliver one or more unit doses of the blocking agent.

For example, the pharmaceutical composition can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. The pharmaceutical composition can be administered with an implant or a module. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

A TWEAK/TWEAK-R blocking agent (e.g., an antibody or soluble TWEAK receptor protein) can be provided in a kit. In one embodiment, the kit includes (a) a container that contains a composition that includes a TWEAK or a TWEAK receptor blocking agent, and optionally (b) informational material. The informational material can be descriptive, instructional, marketing or other material that relates to the methods described herein and/or the use of the agents for therapeutic benefit. In an embodiment, the kit also includes a second agent for treating stroke. For example, the kit includes a first container that contains a composition that includes the TWEAK/TWEAK-R blocking agent, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the TWEAK/TWEAK-R blocking agent (e.g., an antibody or soluble TWEAK receptor protein), e.g., in a suitable dose, dosage form, or mode of administration (e.g., a dose, dosage form, or mode of administration described herein), to treat a subject who has had a stroke or who is at risk for stroke. The information can be provided in a variety of formats, including printed text, computer readable material, video recording, audio recording, or information that provides a link or address to substantive material located on the world wide web.

In addition to the blocking agent, the composition in the kit can include other ingredients, such as a solvent or buffer, a stabilizer, or a preservative. The blocking agent can be provided in any form, e.g., liquid, dried, or lyophilized form, preferably substantially pure and/or sterile. When the agent is provided in a liquid solution, the liquid solution preferably is an aqueous solution. When the agent is provided as a dried form, reconstitution generally is by the addition of a suitable solvent. The solvent, e.g., sterile water or buffer, can optionally be provided in the kit.

The kit can include one or more containers for the composition or compositions containing the agent. In some embodiments, the kit contains separate containers, dividers, or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial, or syringe that has attached thereto the informational material in the form of a label. In some embodiments, the kit includes a plurality (e.g., a pack) of individual containers, each containing one or more unit dosage forms (e.g., a dosage form described herein) of the agent. The containers can include a combination unit dosage, e.g., a unit mat includes both the TWEAK or a TWEAK receptor blocking agent and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the agent (e.g., in a pharmaceutical composition), e.g., a syringe or other suitable delivery device. The device can be provided pre-loaded with one agent or a combination of agents or can be empty, but suitable for loading.

### Nucleic Acid and Protein Analysis

Numerous methods for detecting TWEAK or a TWEAK receptor protein and nucleic acid are available to the skilled artisan, including antibody-based methods for protein detection (e.g., Western blot or ELISA), and hybridization-based methods for nucleic acid detection (e.g., PCR or Northern blot).

Arrays are particularly useful molecular tools for characterizing a sample, e.g., a sample from a subject. For example, an array having capture probes for multiple genes, including probes for TWEAK and a TWEAK receptor, or for multiple proteins, can be used in a method described herein. Arrays can have many addresses, e.g., locatable sites, on a substrate. The featured arrays can be configured in a variety of formats, non-limiting examples of which are described below.

The substrate can be opaque, translucent, or transparent. The addresses can be distributed, on the substrate in one dimension, e.g., a linear array; in two dimensions, e.g., a planar array; or in three dimensions, e.g., a three-dimensional array. The solid substrate may be of any convenient shape or form, e.g., square, rectangular, ovoid, or circular.

Arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT App. No. US/93/04145).

The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the gene. The Tₘ of the capture probe can be optimized by prudent selection of the complementarity region and length. Ideally, the Tₘ of all capture probes on the array is similar, e.g., within 20, 10, 5, 3, or 2° C. of one another.

The isolated nucleic acid is preferably mRNA that can be isolated by routine methods, e.g., including DNase treatment to remove genomic DNA and hybridization to an oligo-dT coupled solid substrate (e.g., as described in Current Protocols in Molecular Biology, John Wiley & Sons, N.Y.). The substrate is washed and the mRNA is eluted.

The isolated mRNA can be reversed transcribed and optionally amplified, e.g., by rtPCR, e.g., as described in U.S. Pat. No. 4,683,202. The nucleic acid can be an amplification product, e.g., from PCR (U.S. Pat. Nos. 4,683,196 and 4,683,202); rolling circle amplification (“RCA,” U.S. Pat. No. 5,714,320), isothermal RNA amplification or NASBA (U.S. Pat. Nos. 5,130,238; 5,409,818; and 5,554,517), and strand displacement amplification (U.S. Pat. No. 5,455,166). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed and then imaged to detect fluorescence at each address of the array.

The expression level of a TWEAK or TWEAK-R protein can be determined using an antibody specific for the polypeptide (e.g., using a Western blot or an ELISA assay). Moreover, the expression levels of multiple proteins, including TWEAK and a TWEAK receptor, can be rapidly determined in parallel using a polypeptide array having antibody capture probes for each of the polypeptides. Antibodies specific for a polypeptide can be generated by a method described herein (see “Antibody Generation”). The expression level of a TWEAK or TWEAK receptor can be measured in a subject (e.g., in vivo imaging) or in a biological sample from a subject (e.g., blood, serum, plasma, or cerebral spinal fluid).

A low-density (96-well format) protein array has been developed in which proteins are spotted onto a nitrocellulose membrane (Ge (2000) Nucleic Acids Res. 28, e3, I-VII). A high-density protein array (100,000 samples within 222×222 mm) used for antibody screening was formed by spotting proteins onto polyvinylidene difluoride (PVDF) (Lueking et al. (1999) Anal. Biochem. 270:103-111). See also, e.g., Mendoza et al. (1999) Biotechniques 27:778-788; MacBeath and Schreiber (2000) Science 289:1760-1763; and De Wildt et al. (2000) Nat. Biotech. 18:989-994. These art-known methods and others can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address.

The nucleic acid and polypeptide arrays of the invention can be used in a wide variety of applications. For example, the arrays can be used to analyze a patient sample. The sample is compared to data obtained previously, e.g., known clinical specimens or other patient samples. Further, the arrays can be used to characterize a cell culture sample, e.g., to determine a cellular state after varying a parameter, e.g., exposing the cell culture to an antigen, a transgene, or a test compound.

The expression data can be stored in a database, e.g., a relational database such as a SQL database (e.g., Oracle or Sybase database environments). The database can have multiple tables. For example, raw expression data can be stored in one table, wherein each column corresponds to a gene being assayed, e.g., an address or an array, and each row corresponds to a sample. A separate table can store identifiers and sample information, e.g., the batch number of the array used, date, and other quality control information.

Expression profiles obtained from gene expression analysis on an array can be used to compare samples and/or cells in a variety of states as described in Golub et al. ((1999) Science 286:531). In one embodiment, expression (e.g., mRNA expression or protein expression) information for a gene encoding TWEAK and/or a gene encoding a TWEAK receptor are evaluated, e.g., by comparison to a value, e.g., a reference value. Reference values can be obtained from a control, e.g., a reference subject. Reference values can also be obtained from statistical analysis, e.g., to provide a reference value for a cohort of subjects, e.g., age and gender matched subjects, e.g., normal subjects or subjects who have sustained a stroke. Statistical similarity to a particular reference (e.g., to a reference for a risk-associated cohort) or a normal cohort can be used to provide an assessment (e.g., an indication of stroke risk) to a subject, e.g., a subject who has not sustained a prior stroke, a subject who has sustained a TIA, or a subject who has sustained a stroke.

Subjects suitable for treatment can also be evaluated for expression and/or activity of TWEAK and/or a TWEAK receptor. Subjects can be identified as suitable for treatment if the expression and/or activity for TWEAK and/or a TWEAK receptor is elevated relative to a reference, e.g., reference value, e.g., a reference value associated with normal.

Subjects who are being administered an agent described herein or other stroke treatment can be evaluated as described for expression and/or activity of TWEAK and/or a TWEAK receptor. The subject can be evaluated at multiple times, e.g., at multiple times during a course of therapy, e.g., during a therapeutic regimen. Treatment of the subject can be modified depending on how the subject is responding to the therapy. For example, a reduction in TWEAK and/or TWEAK receptor expression or activity can be indicative of responsiveness.

Particular effects mediated by an agent may show a difference (e.g., relative to an untreated subject, control subject, or other reference) that is statistically significant (e.g., P value < 0.05 or 0.02). Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02.

### In Vivo Imaging

TWEAK and/or TWEAK receptor blocking agents (e.g., antibodies) provide a method for detecting the presence of TWEAK and/or a TWEAK receptor (e.g., Fn14) in vivo (e.g., in vivo imaging in a subject), respectively. The method can be used to evaluate (e.g., diagnose, localize, or stage) a condition described herein, e.g., a stroke or risk of stroke. The method includes: (i) administering to a subject (and optionally a control subject) a TWEAK or TWEAK-R binding agent (e.g., a blocking agent that binds to TWEAK or a TWEAK receptor, e.g., an antibody or antigen-binding fragment thereof, although such agents need not be blocking agents), under conditions that allow interaction of the binding agent and TWEAK or TWEAK receptor to occur; and (ii) detecting localization of the binding agent in the subject. The method can be used to detect the location of TWEAK or TWEAK-R expressing cells. A statistically-significant increase in the amount of the complex in the subject relative to the reference, e.g., the control subject or subject's baseline, can be a factor that may lead to a diagnosis of stroke or risk for stroke.

Preferably, the TWEAK and/or TWEAK-R binding agent used in the in vivo (and also in vitro) diagnostic methods is directly or indirectly labeled with a detectable substance to facilitate detection of the bound or unbound binding agent. Suitable detectable substances include various enzymes, prosthetic groups, fluorescent materials, luminescent materials and radioactive materials. In one embodiment, the TWEAK or TWEAK-R binding protein is coupled to a radioactive ion, e.g., indium (¹¹¹In), iodine (¹³¹I or ¹²⁵I), yttrium (⁹⁰Y), actinium (²²⁵Ac), bismuth (²¹²Bi or ²¹³Bi), sulfur (³⁵S), carbon (¹⁴C), tritium (³H), rhodium (¹⁸⁸Rh), or phosphorous (³²P). In another embodiment, the TWEAK/TWEAK-R binding protein is labeled with an NMR contrast agent.

In one aspect, the invention features a method of imaging vasculature in a patient who is at risk for stroke, has experienced a stroke, and/or is recovering from a stroke. The method includes: providing an agent that binds to TWEAK or a TWEAK receptor, e.g., an agent described herein, wherein the protein is physically associated to an imaging agent; administering the agent to a patient, e.g., with a risk for stroke; and imaging the patient, e.g., to detect TWEAK or TWEAK receptor expressing cells.

### Methods of Evaluating Genetic Material

There are numerous methods for evaluating genetic material to provide genetic information. These methods can be used to evaluate a genetic locus that includes a gene encoding TWEAK or a gene encoding a TWEAK receptor, as well as other loci. The methods can be used to evaluate one or more nucleotides, e.g., a coding or non-coding region of the gene, e.g., in a regulatory region (e.g., a promoter, a region encoding an untranslated region or intron, and so forth).

Nucleic acid samples can be analyzed using biophysical techniques (e.g., hybridization, electrophoresis, and so forth), sequencing, enzyme-based techniques, and combinations thereof. For example, hybridization of sample nucleic acids to nucleic acid microarrays can be used to evaluate sequences in an mRNA population and to evaluate genetic polymorphisms. Other hybridization-based techniques include sequence specific primer binding (e.g., PCR or LCR); Southern analysis of DNA, e.g., genomic DNA; Northern analysis of RNA, e.g., mRNA; fluorescent probe-based techniques (see, e.g., Beaudet et al. (2001) Genome Res. 11(4):600-608); and allele specific amplification. Enzymatic techniques include restriction enzyme digestion; sequencing; and single base extension (SBE). These and other techniques are well known to those skilled in the art.

Electrophoretic techniques include capillary electrophoresis and Single-Strand Conformation Polymorphism (SSCP) detection (see, e.g., Myers et al. (1985) Nature 313:495-498 and Ganguly (2002) Hum. Mutat. 19(4):334-342). Other biophysical methods include denaturing high pressure liquid chromatography (DHPLC).

In one embodiment, allele specific amplification technology that depends on selective PCR amplification may be used to obtain genetic information. Oligonucleotides used as primers for specific amplification may carry the mutation of interest in the center of the molecule (so that amplification depends on differential hybridization) (Gibbs et al. (1989) Nucl. Acids Res. 17:2437-2448) or at the extreme 3′ end of one primer where, under appropriate conditions, mismatch can prevent, or reduce, polymerase extension (Prosser (1993) Trends Biotechnol. 11:238-246). In addition, it is possible to introduce a restriction site in the region of the mutation to create cleavage-based detection (Gasparini et al. (1992) Mol. Cell. Probes 6:1). In another embodiment, amplification can be performed using Taq ligase for amplification (Barany (1991) Proc. Natl. Acad. Sci. USA 88:189). In such cases, ligation will occur only if there is a perfect match at the 3′ end of the 5′ sequence, making it possible to detect the presence of a known mutation at a specific site by looking for the presence or absence of amplification.

Enzymatic methods for detecting sequences include amplification-based methods such as the polymerase chain reaction (PCR; Saiki et al. (1985) Science 230:1350-1354) and ligase chain reaction (LCR; Wu et al. (1989) Genomics 4:560-569; Barringer et al. (1990) Gene 1989:117-122; F. Barany (1991) Proc. Natl. Acad. Sci. USA 1988:189-193); transcription-based methods utilizing RNA synthesis by RNA polymerases to amplify nucleic acid (U.S. Pat. Nos. 6,066,457; 6,132,997; and 5,716,785; Sarkar et al. (1989) Science 244:331-334; Stofler et al. (1988) Science 239:491); NASBA (U.S. Pat. Nos. 5,130,238; 5,409,818; and 5,554,517); rolling circle amplification (RCA; U.S. Pat. Nos. 5,854,033 and 6,143,495), and strand displacement amplification (SDA; U.S. Pat. Nos. 5,455,166 and 5,624,825). Amplification methods can be used in combination with other techniques.

Other enzymatic techniques include sequencing using polymerases, e.g., DNA polymerases and variations thereof, such as single base extension technology. See, e.g., U.S. Pat. Nos. 6,294,336; 6,013,431; and 5,952,174.

Fluorescence-based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (e.g., “color”) of the fluorescent dye that is incorporated.

Hybridization to microarrays can also be used to detect polymorphisms, including SNPs. For example, a set of different oligonucleotides, with the polymorphic nucleotide at varying positions within the oligonucleotides, can be positioned on a nucleic acid array. The extent of hybridization as a function of position and hybridization to oligonucleotides specific for the other allele can be used to determine whether a particular polymorphism is present. See, e.g., U.S. Pat. No. 6,066,454.

In one implementation, hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other.

It is also possible to directly sequence the nucleic acid for a particular genetic locus, e.g., by amplification and sequencing, or amplification, cloning, and sequencing. High throughput automated (e.g., capillary or microchip based) sequencing apparati can be used. In still other embodiments, the sequence of a protein of interest is analyzed to infer its genetic sequence. Methods of analyzing a protein sequence include protein sequencing, mass spectroscopy, sequence/epitope specific immunoglobulins, and protease digestion.

Any combination of the above methods can also be used. The above methods can be used to evaluate any genetic locus, e.g., in a method for analyzing genetic information from particular groups of individuals or in a method for analyzing a polymorphism associated with stroke, e.g., in a gene encoding TWEAK or a TWEAK receptor R.

### EXAMPLES

TWEAK and Fn14 are upregulated in in vivo and in vitro models of cerebral ischemia. A neutralizing anti-TWEAK antibody reduced the infarct size in a model of permanent cerebral ischemia. Compared with transient cerebral ischemia, models of permanent cerebral ischemia provide a more stringent test of neuroprotection (Chan et al. (1993) Neuroreport 5:293-296; Lou et al. (2004) Stroke 35:578-583). Even at the center of the ischemic region there is residual blood flow (Love (2003) Prog. Neuropsychopharmacol. Biol. Psychiatry 27:267-282). Thus, the antibody can access the ischemic brain through a permeable blood-brain barrier.

In vivo neutralization of TWEAK may protect against stroke-induced ischemic brain damage not only by preventing neuronal cell death, but also by reducing neuroinflammation, gliosis, and scarring that result as a consequence of stroke.

Materials and Methods

Materials. The production of recombinant soluble human TWEAK (rhTWEAK), containing amino-acid residues A106-H249, has been described previously (Jakubowski et al. (2002) J. Cell Sci. 115:267-274). For Fc-hTWEAK, a plasmid was generated that contains the sequences corresponding to a human IgG1 Fc fragment (aa 108-338 of GenBank accession number AAC82527 excluding the stop codon), a linker sequence, and the receptor-binding domain of TWEAK (aa 106-249). The construct was stably transfected into the 293T cell line. Fc-hTWEAK was purified with protein A. Anti-Fn14 serum was generated by immunizing Fn14 knock-out (KO) mice with purified recombinant murine Fn14 (aa 28-79) containing a myc-His tag at the C-terminal end. Anti-Fn14 monoclonal antibody 1.P1C12.1D8 was generated using the above immunized Fn14 KO mice as described previously (Kennett et al. (1982) Monoclonal Antibodies. A new dimension in biological analysis. New York:Plenum). SN50 was purchased from Biomol (Hamburg, Germany), murine TNF-α from Sigma (Munich, Germany), and ITEM-4 from eBioscience (San Diego, Calif.; Cat. No. 12-9018).

MPSS expression profiling. For expression profiling, the filament model of middle cerebral artery occlusion (MCAO) was used. Mice (129X1/SV^{J}) were anesthetized using 70% N₂O, 30% O₂, and 1% halothane. A 5-0 nylon filament blunted at the tip was inserted into the common carotid artery. The filament was advanced into the internal carotid artery until the middle cerebral artery was reached. Successful occlusion was monitored using laser Doppler flowmetry (Perimed, Stockholm, Sweden). The 90 minutes occlusion was followed by a 20 hours reperfusion. Thereafter, animals were killed under deep anesthesia by transcardial perfusion with HBSS. Hemispheric forebrains (cerebellum, olfactory bulb, and brainstem removed) were further processed for RNA using acidic phenol extraction. For expression profiling, RNA from hemispheres (ipsilateral and contralateral) of six animals was pooled to reduce the influence of interindividual variation in infarct severity. Results from two MPSS runs per sample were pooled.

For MPSS, RNA was converted into cDNA and the most 3′ DpnII fragments were recovered. After in vitro cloning, the cDNA templates were immobilized on separate glass beads of 5 μm diameter. Loaded microbeads were placed into a flow cell forming a densely packed monolayer. Short sequences from the free template ends were obtained simultaneously by a fluorescence-based ligation-mediated sequencing method. Obtained signatures (14 bases) were sufficiently long to allow the identification of the vast majority (> 95%) (cf. Velculescu et al. (1995) Science 270:484-487) of the individual cDNAs. Signatures matching more than one gene (taking into account only nonexpressed sequence tags or expressed sequence tag European Molecular Biology Laboratory database entries) could be detected by clustering all matching sequences, excluding putative sequencing errors and sequence polymorphisms.

To prepare cDNA libraries for the MPSS analysis, 5 μg of oligo-dT-cellulose (Peqlab, Erlangen, Germany)-enriched A+RNA was denatured at 70° C. with 50 pmol of BsmBI-oligo-dT18V primer (for further details, see Potrovita et al. (2004) J. Neurosci. 24:8237-8244), cooled on ice, and reverse-transcribed with 200 U of Superscript™ II at 42° C. for 1 hour in 1× reaction buffer, 10 mM dithiothreitol (all reagents from Invitrogen, Karlsruhe, Germany), and 0.5 mM each dNTP (Roche Diagnostics, Mannheim, Germany) in 25 μl. Second-strand cDNA synthesis was performed by adding 40 U of DNA polymerase I, 2 U of RNase H, 10 U of Escherichia coli DNA ligase, and 0.5 mM each dNTP in 1× second-strand buffer (Invitrogen) in a final volume of 100 μl for 2 hours at 16° C. RNA was hydrolyzed in the presence of 100 mM NaOH at 65° C. for 20 minutes. The reaction product was phenol/chloroform-purified and precipitated with ammonium acetate in the presence of PELLETPAINT® (Calbiochem, La Jolla, Calif.; Novabiochem, Bad Soden, Germany).

Resuspended, double-stranded cDNA was digested with DpnII; 3'DpnII fragments were isolated with streptavidin-coupled paramagnetic beads (Dynal Biotech, Hamburg, Germany) and released from the beads with a BsmBI digest. DpnII-BsmBI double-stranded cDNA fragments were tagged by cloning the fragments into the TAG vector (pLCV), which was digested with BbsI and BamHI. After electroporation of DH10B E. coli (Invitrogen) for each sample, 106 independent clones were harvested, and the plasmid DNA containing the tagged cDNA was extracted. Using PCR, the tagged cDNAs were amplified from the plasmid DNA and mixed with microbeads (LYNX, Hayward, Calif.) carrying the complementary antitags. The tagged cDNA was loaded onto the microbeads by hybridizing the tags to the antitags. The DNA-loaded beads were loaded into a flow cell and further processed on an MPSS instrument (LYNX) as described (Brenner et al. (2000) Nat. Biotechnol. 18:630-634).

Cell culture and transient transfection. Cortical neurons were prepared from embryonic day 16 (E16) mice. For transfection, cells were plated on 24-well plates precoated with poly-D-lysine (50 ng/ml) at a density of 200,000 cells per well. For RNA preparation, 2 million cells per well were plated on six-well plates. Cells were incubated in NEUROBASAL™ medium (Invitrogen) supplemented with B27 (Invitrogen), L-glutamine (0.5 mM), penicillin (100 IU/ml), and streptomycin (100 μg/ml). In these cultures, >95% of cells were positive for the neuronal marker NeuN. After 10 days in vitro, cells were transfected using LIPOFECTAMINE™ 2000 (Invitrogen) and 1 ng per well of the NF-κB reporter plasmid pNF-κB-Luc, which has five tandem repeats of an NF-κB binding site (Stratagene, Amsterdam, The Netherlands), according to the manufacturer's protocol. After 24 hours, cells were stimulated as indicated and harvested. Luciferase activity was measured as described (Sallmann et al. (2000) J. Neurosci. 20:8637-8642).

As indicated, cortical neurons were prepared from a transgenic mouse line (NSE-IκBα-SR) that expressed the NF-κB super-repressor, a mutant of IκBα (IκBα-SR), selectively in neurons. In this transgene, two serine residues, which are phosphorylated by the IκB kinase (IKK), are exchanged to alanines. Neuronal expression is driven by a 1.8 KB fragment of the rat neuron-specific enolase (NSE) promoter. The cell dissociation was performed individually from the brain of each transgenic or wild-type embryo. To control for transfection efficiency, cortical neurons of NSE-IκBα-SR mice and wild-type littermates were cotransfected with 0.1 μg Per well of phRL-TK (Promega, Mannheim, Germany) in addition to pNF-κB-Luc.

Cell death assays. For terminal deoxynucleotidyl transferase-mediated biotinylated UTP nick end labeling (TUNEL) staining, the cells were fixed in 4% paraformaldehyde at room temperature for 30 minutes. Then, cells were washed twice in PBS for 5 minutes and treated for 2 minutes with 200 μl of permeabilization solution (0.1% Triton X-100 and 0.1% sodium citrate in PBS) at 4° C. After washing, sections were incubated with 50 μl of TUNEL reaction mix (enzyme solution diluted 1:6 in labeling solution; In Situ Cell Detection Kit, Fluorescein; Roche, Mannheim, Germany) for 1 hour at 37° C. in the dark. Then, coverslips were mounted with medium containing 4′,6-diamidino-2-phenylindole dihydrochloride (DAPI; VECTASHIELD®; Vector Laboratories, Burlingame, Calif.). TUNEL and DAPI-positive cells were detected under a fluorescent microscope. On each coverslip, five randomly selected fields were counted with a 40× objective (corresponding to ˜800 cells per coverslip). Cell Death Detection ELISA^{Plus} (Roche) was performed according to the manufacturer's instructions. Lactate dehydrogenase (LDH) activity in medium was quantified with the Cytotoxicity Detection Kit (LDH; Roche). LDH activity in untreated sister cultures was subtracted, and the LDH activity was expressed as percentage of the maximally releasable LDH pool in the presence of 1 mM glutamate.

In situ hybridization and immunocytochemistry. Techniques for in situ analysis have been described in detail previously (Sassoon et al. (1988) Development 104:155-164; Sassoon and Rosenthal (1993) Methods Enzymol. 225:384-404). Fn14 riboprobe templates were generated using specific PCR primers containing a T7 polymerase promoter binding site on either the forward or reverse primer to generate sense or antisense templates, respectively. The 448 bp riboprobe template is specific for nucleotides 9-456 in the murine Fn14 coding domain (GenBank accession number BC025860). High specific-activity probes were synthesized using an AMBION T7 MAXISCRIPT® in vitro transcription kit and ³³P-radiolabeled UTP (> 3000 Ci/mmol; PerkinElmer Life Sciences, Wellesley, Mass.), according to the manufacturer's instructions. All probes were used in a hybridization buffer containing 30,000 counts per minute per milliliter final probe concentration. Microscopic analysis of the expression patterns was performed on a LEICA DMR™ system modified for reflective dark-field microscopy. Images were captured using a COOLSNAP™ RGB camera, processed in OPEN LAB®, and polished using PHOTOSHOP® 7.0.

For immunohistochemistry, cells were fixed for 5 minutes in acetone at room temperature. Then, cells were dried for 30 minutes and rehydrated for 5 minutes in PBS. After 1 hour of blocking with 5% horse serum, polyclonal mouse anti-Fn14 or preimmune sera (1:300 dilution) and culture supernatant containing anti-Fn14 monoclonal antibody (mAb) 1.P1C12.1D8 or DMEM media as control were added and left to stand overnight at 4° C. After washing three times in PBS, biotinylated horse anti-mouse IgG (diluted 1:200; Vector) and FITC-avidin (1:200 dilution; Vector) were used for detection. Coverslips were mounted with medium containing DAPI. Cells were analyzed under a fluorescent microscope.

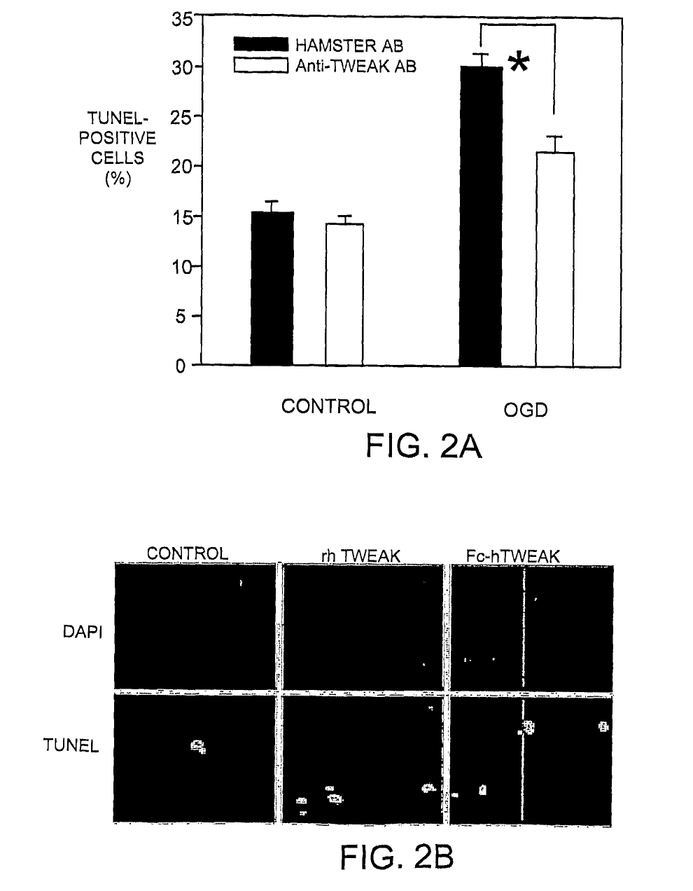

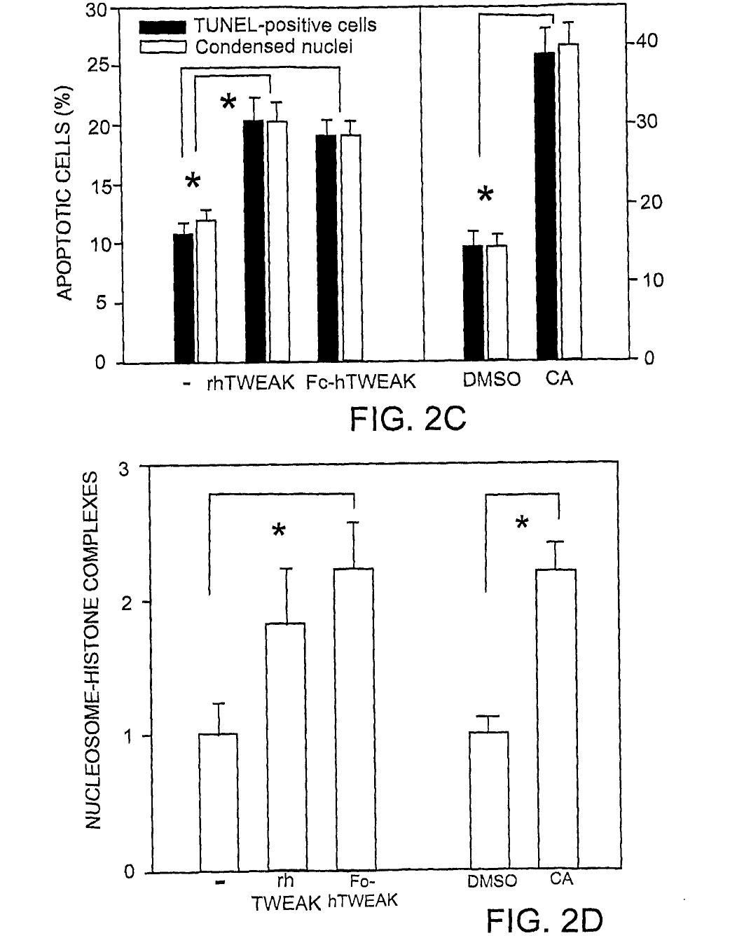

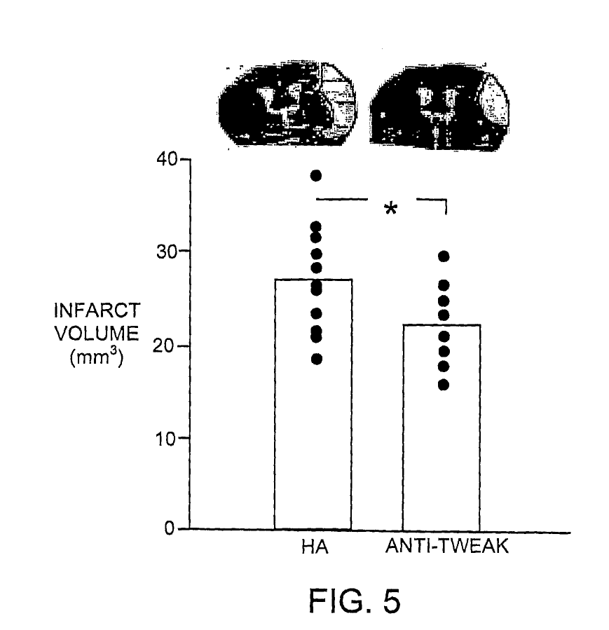

Models of cerebral ischemia. As an vivo model of permanent focal cerebral ischemia, a distal MCAO was performed (see FIGS. 2 and 5). At an age of 3-4 months, male C57BL/6 mice were anesthetized by intraperitoneal injection of 150 μl of 2.5% avertin (tribromoethanol) per 10 gm of body weight. A skin incision was made between the ear and the orbit on the left side. The parotid gland and the temporal muscle were removed by electrical coagulation. The stem of the MCA was exposed through a bun-hole and occluded by microbipolar coagulation (Erbe, Tübingen, Germany). Surgery was performed under a microscope (Hund, Wetzlar, Germany). Mice were kept at a body temperature of 37° C. on a heating pad. The anti-TWEAK antibody AB.G11 (Jakubowski et al. (2002) J. Cell Sci. 115:267-274) (200 μg) or the same amount of an unspecific hamster Ig (Id.) was injected intraperitoneally 10 minutes before MCAO. After 48 hours mice were deeply re-anesthetized with AVERTIN® and perfused intracardially with Ringer's solution. The procedure for infarct measurement on cryo-sections and correction for cerebral edema has been described previously (Herrmann et al. (2003) J. Cereb. Blood Flow Metab. 23:406-415). Surgery was performed and infarcts measured without knowledge of the treatment group. In a separate cohort of animals, the femoral artery was cannulated for measurement of arterial blood gases and mean arterial blood pressure. Arterial blood gases, glucose, and hemoglobin were measured immediately before and 15 minutes into MCAO in a blood sample of 100 μl. For laser Doppler measurements, the probe (P415-205; Perimed) was placed 3 mm lateral and 6 mm posterior to the bregma. Relative perfusion units were determined (Periflux 4001; Perimed).

Oxygen glucose deprivation (OGD) was used as an in vitro model of ischemia. For OGD experiments, primary cortical neurons, which had been in culture for 10 days, were transferred into serum-free medium containing 5 mM 2-deoxy-D-glucose (Merck, Darmstadt, Germany) for 1 hour. Then, the cells were placed in an anaerobic chamber that was flushed for 10 minutes with a mix of 95% N₂ and 5% CO₂. After incubation for the indicated times, cells were removed from the anaerobic chamber and incubated under normal conditions for another 24 hours. The control group was cultured in parallel but did not receive 2-deoxy-D-glucose and was not flushed with N₂/CO₂. Then, RNA was extracted.

Real-time RT-PCR. Mice were re-anesthetized and perfused with Ringer's solution 24 hours after MCAO. The ischemic and contralateral cortices were quickly dissected and frozen on dry ice. Tissues were stored at −80° C. RNA from cortex or cultured cells was extracted with PEQGOLD RNAPURE™ (PEQLAB, Erlangen, Germany), according to the manufacturer's instructions. RNA (10 μg, cortex; 7.5 μg, cells) was transcribed with Moloney murine leukemia virus reverse transcriptase and random hexamers. The primers used for PCR amplification are described in Potrovita et al., (2004). PCR was performed according to the following protocol: 10 minutes at 95° C., 15 seconds at 95° C., and 1 minute at 60° C. (40 cycles). Amplification was quantified with the Gene Amp 5700 sequence detector and the SYBR® Green kit (PE Diagnostik, Weiterstadt, Germany). A linear concentration-amplification curve was established by diluting pooled samples. Quantified results for individual cDNAs were normalized to cyclophilin. This procedure allows us to quantify results relative to a control group. The purity of the amplified products was checked by the dissociation curve.

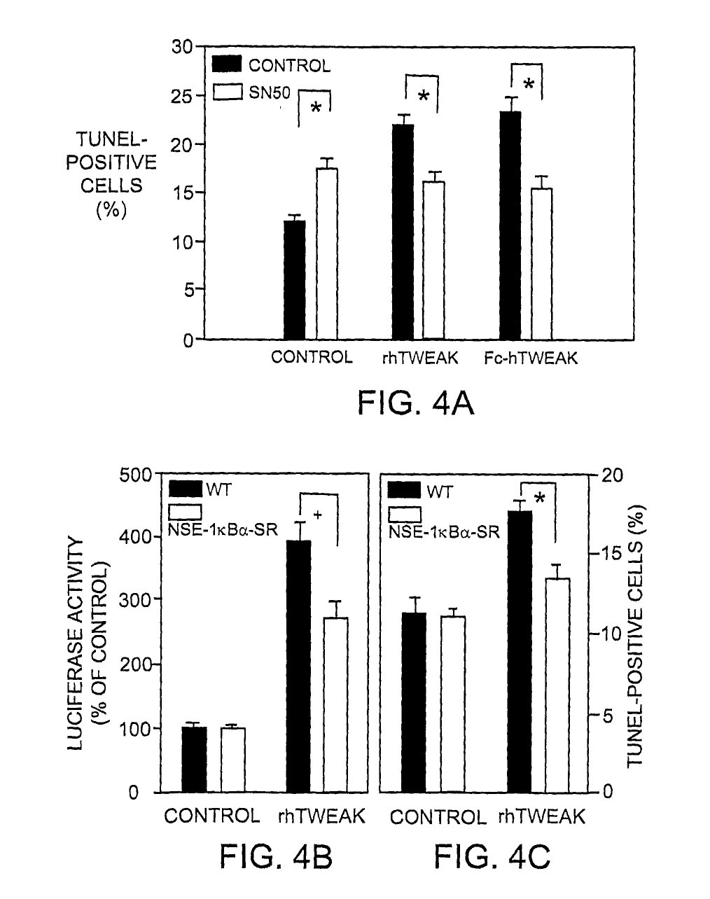

Statistical analysis. Data are presented as mean±SE. Statistical comparisons of three or more groups were made by ANOVA followed post hoc by Fisher's protected least-squares difference (LSD). Two groups were compared by a two-sided t test or by two-sided Mann-Whitney U test in the case of counted data (see FIGS. 2A, 4C). Values were considered significant at p< 0.05. In MPSS, the statistical significance of the observed signature frequency distributions was calculated according to Equation 2 of Audio and Claverie (1997) Genome Res. 7:986-995. p< 0.001 was considered significant.

### Example 1

### TWEAK and Fn14 mRNA are Upregulated By Cerebral Ischemia

Results of two MPSS runs performed with RNA from the ipsilateral forebrain of mice subjected to MCAO versus contralateral forebrain (after 90 minutes occlusion and 20 hours reperfusion) yielded a total of 174,812 tag sequences for the contralateral and 161,809 tag sequences for the ischemic hemispheres. Identical sequences were clustered to display the expression levels of the corresponding genes, resulting in 58,315 different clusters and including sequences with ambiguities (one or more nucleotides not fully determined). Subsequently, the number of occurrences of individual signatures (pool of two replicate runs) as a direct measure of expression level was compared between the two experimental samples. One of the signatures that was induced had a tag frequency of 41 in the ipsilateral versus 0 in the contralateral hemisphere (p˜10⁻¹²). A database search identified this signature as corresponding to mouse TWEAK mRNA (GENBANK® accession number AF030100).

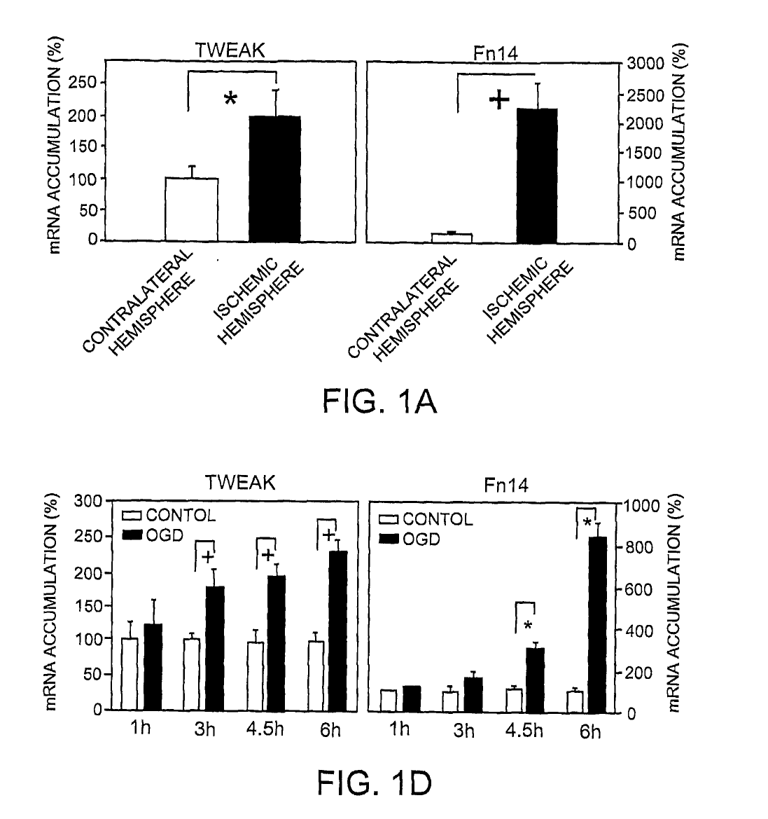

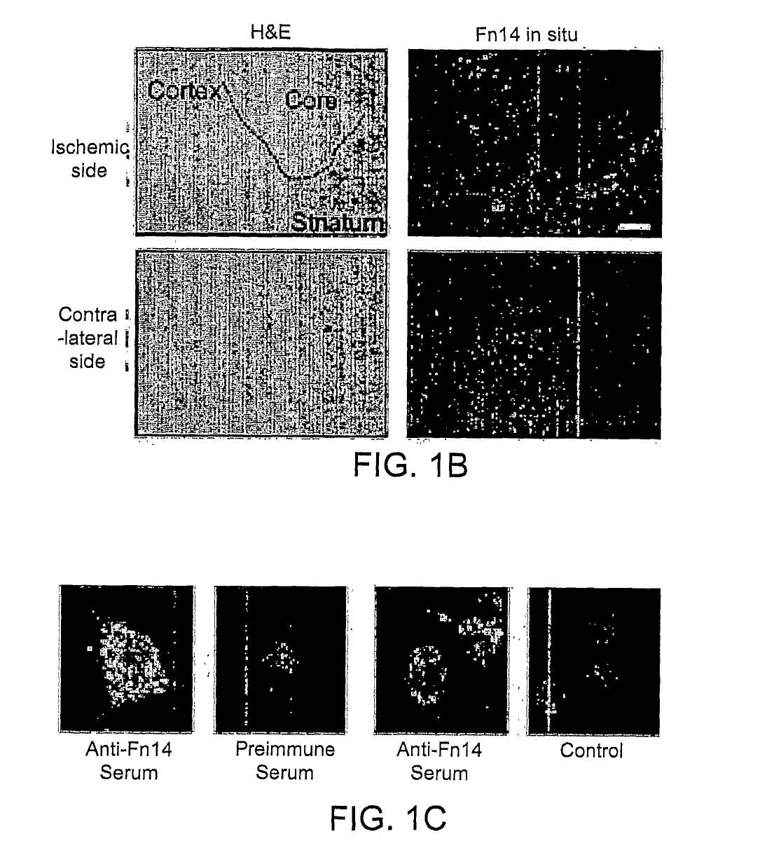

Induction of TWEAK mRNA by cerebral ischemia was verified by RT-real-time PCR. TWEAK was upregulated more than three-fold in the ischemic as compared with the contralateral hemisphere (data not shown). In another stroke model, distal MCAO, there was a 2.0-fold increase in TWEAK mRNA in the ischemic as compared with the contralateral hemisphere 24 hours after occlusion onset (FIG. 1A, left) (p<0.02). Induction of the TWEAK receptor Fn14, however, was more pronounced. Quantification of Fn14 mRNA by RT-real-time PCR revealed a 22.3-fold upregulation after 24 hours of MCAO compared with the contralateral cortex (FIG. 2A, right). By in situ hybridization, elevated levels of Fn14 transcripts were found in the periphery of the cortical ischemia, the putative penumbra (FIG. 1B).

Fn14 has been shown to be expressed by peripheral neurons (Tanabe et al. (2003) J. Neurosci. 23:9675-9686), and the in situ hybridization signals were compatible with a predominantly neuronal origin of the Fn14 upregulation in cerebral ischemia. Accordingly, cortical neurons expressed Fn14 in vitro, as demonstrated by immunocytochemistry with anti-Fn14 serum and an Fn14-specific mAb ((FIG. 1C). Furthermore, the mRNA could be detected by RT-real-time PCR (FIG. 1D, right). In cortical neurons, OGD, an in vitro model of cerebral ischemia, induced a marked upregulation of Fn14 after 4.5 and 6 hours (FIG. 1D, right). TWEAK mRNA in neurons was also upregulated by OGD, but the induction occurred at 3 hours and was less pronounced (FIG. 1D left).

### Example 2

### Inhibition of TWEAK Decreases Apoptosis in Cerebral Ischemia

To explore the functional effect of TWEAK upregulation after OGD, a neutralizing monoclonal hamster anti-TWEAK antibody, AB.G11 was used (Jakubowski et al. (2002) J. Cell Sci. 115:267-274), and neuronal apoptosis was quantified by counting TUNEL-positive cells 24 hours after OGD. Induction of apoptosis by 4.5 hours of OGD was clearly ameliorated by inhibition of TWEAK (FIG. 2A). After 4.5 hours of OGD, the percentage of TUNEL-positive cells of ˜30% represented only a part of neuronal cell death. In the same experiments, LDH release as a measure of necrotic cell death increased to 69.2±6.9% of the maximally releasable LDH pool (n=18). The anti-TWEAK antibody had only a slight, insignificant effect on LDH release (data not shown). To investigate whether exogenous TWEAK is able to induce neuronal cell death, rhTWEAK or the fusion protein Fc-hTWEAK was used. Human TWEAK has been shown to bind to murine cells (Jakubowski et al., 2002). Fc-hTWEAK or rhTWEAK exposure for 24 hours induced nuclear condensation as shown by a DAPI stain. In addition, the number of cortical neurons that were TUNEL positive increased significantly after exposure to rhTWEAK or Fc-hTWEAK (FIGS. 2B, C). An ELISA measuring DNA-histone complexes specific to apoptotic DNA fragmentation supported the notion of programmed cell death being induced by rhTWEAK and Fc-hTWEAK (FIG. 2D). The effect size was comparable with the one with 10 μM camptothecin, a classic inducer of apoptosis by DNA damage (FIG. 2D).

### Example 3

### TWEAK Signaling via Fn14 Activates NF-κB

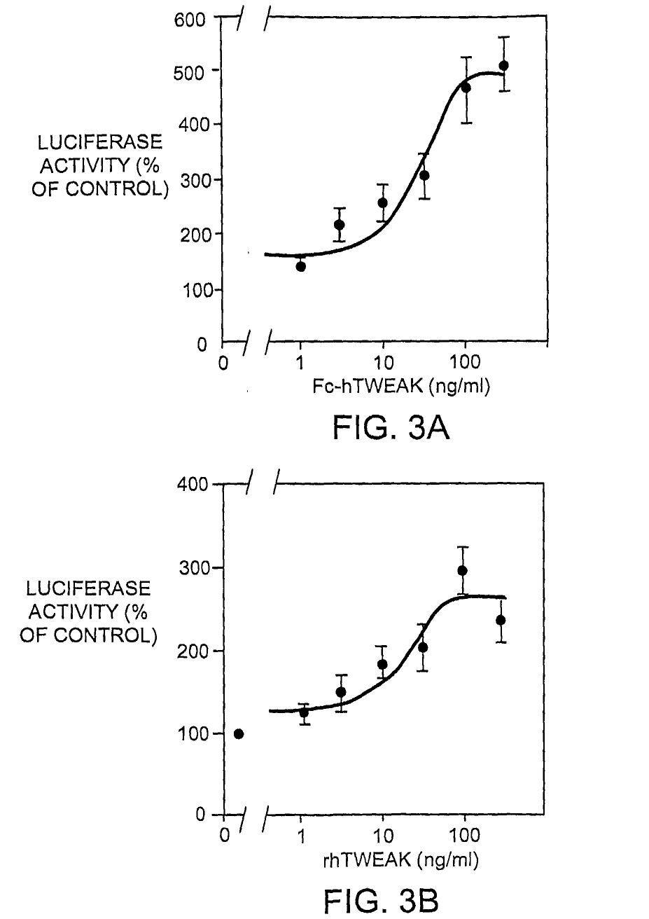

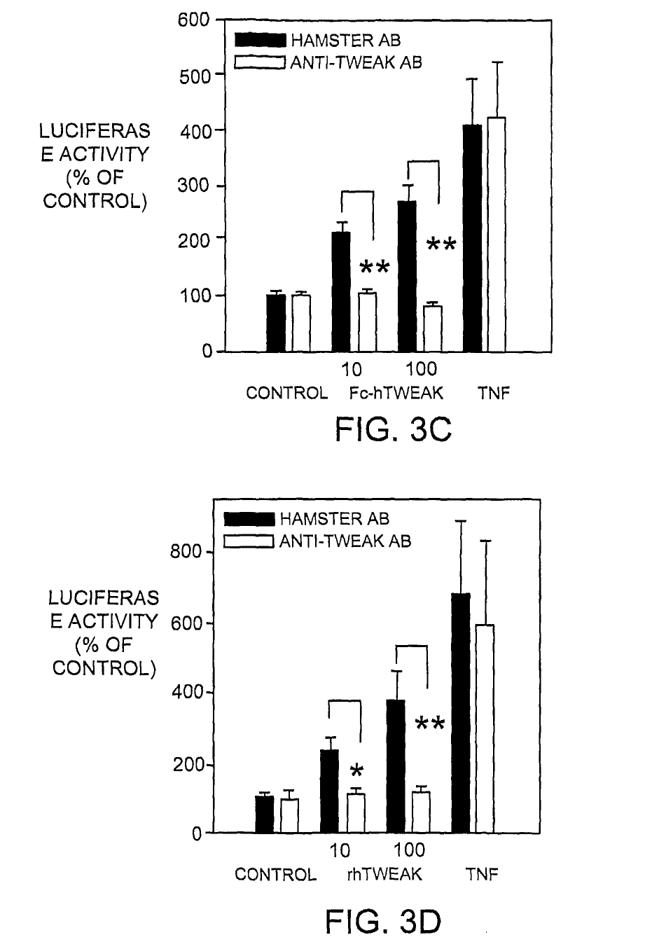

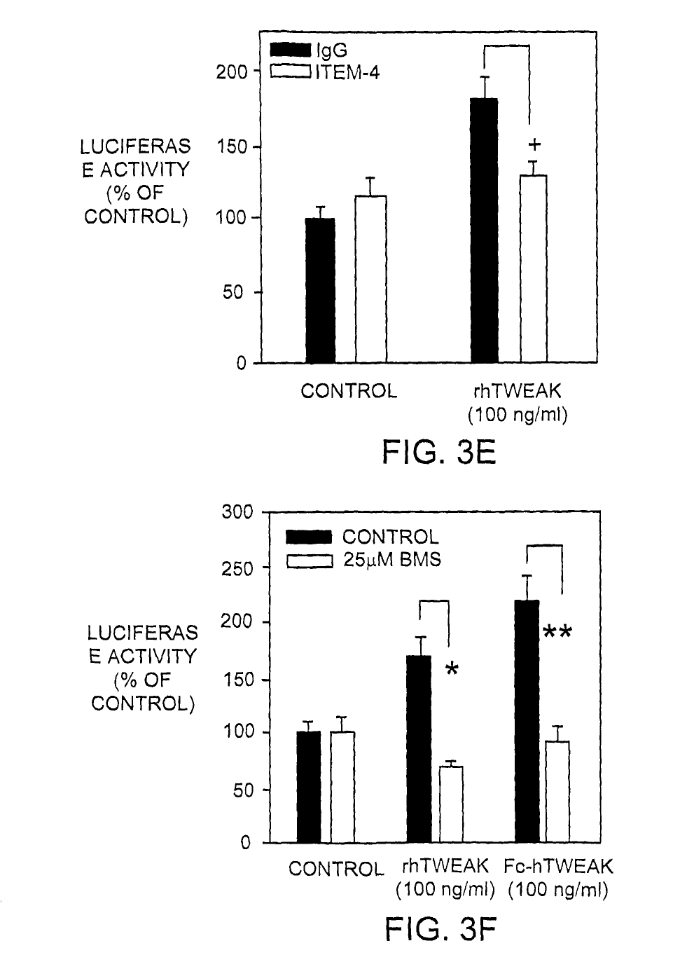

Fn14 does not contain a death domain but binds TNF receptor-associated factors (Wiley et al. (2001) Immunity 15:837-846; Brown et al. (2003) Biochem. J. 371:395-403) that are known to link receptors of the TNF receptor superfamily to several signal transduction pathways. Indeed, TWEAK activates the transcription factor NF-κB via Fn14 (Brown et al., 2003). Because NF-κB has important functions in determining neuronal death or survival (Mattson and Camandola (2001) J. Clin. Invest. 107:247-254), the question of whether TWEAK also activates NF-κB in cortical neurons was addressed. Primary cortical neurons were transfected with a luciferase fusion gene that is under transcriptional control of five NF-κB binding sites. Fc-hTWEAK and rhTWEAK stimulated NF-κB activity in a concentration-dependent manner (FIG. 3D). The effect size varied between experimental series, and there was no consistent difference between Fc-hTWEAK and rhTWEAK (FIGS. 3A,B). The neutralizing anti-TWEAK antibody AB.G11 abrogated NF-κB stimulation by Fc-hTWEAK and rhTWEAK in cortical neurons but had no effect on the stimulation by TNF-α, demonstrating that the effect of TWEAK cytokine stimulation was specific (FIGS. 3C, D). Fn14 mediates many effects of TWEAK, but there is evidence for a second, still unknown TWEAK receptor (Polek et al. (2003) J. Biol. Chem. 278:32317-32323). To test whether Fn14 is responsible for NF-κB stimulation in cortical neurons, the anti-Fn14 antibody ITEM-4, which functions as a competitive TWEAK antagonist at the Fn14 receptor and possesses only a low intrinsic agonist activity, was used (Nakayama et al. (2003) J. Immunol. 170:341-348). When administered alone, ITEM-4 stimulated NF-κB activity slightly, but it inhibited the stimulation by TWEAK in accordance with its function as an antagonist of Fn14 (FIG. 3E). In the classic signaling cascade of NF-κB activation, the IKK plays a pivotal role (Li and Verma (2002) Nat. Rev. Immunol. 2:725-734). To test for the involvement of IKK in NF-κB activation by TWEAK, BMS-345541, a highly specific inhibitor of the IκB kinase, was used (Burke et al. (2003) J. Biol. Chem. 278:1450-1456). BMS-345541 (25 μM) blocked NF-κB activation by rhTWEAK and Fc-hTWEAK, showing that TWEAK stimulates NF-κB via the classic, IKK-dependent pathway in neuronal cells (FIG. 3F).

### Example 4

### NF-κB Mediates TWEAK-Induced Apoptosis

To examine the role of NF-κB in TWEAK-induced neuronal cell death, its activation was inhibited by SN50. In a concentration of 10 μg/ml, SN50 partially inhibited NF-κB activated by rhTWEAK (data not shown). SN50 itself had toxic effects on neurons; however, it significantly reduced the pro-apoptotic effect of rhTWEAK and Fc-hTWEAK (FIG. 4A). rhTWEAK stimulated NF-κB significantly less in cortical neurons of NSE-IκBα-SR mice than in those of wild-type littermates (FIG. 4B). In parallel to the reduced NF-κB stimulation, TWEAK-induced neuronal cell death was also significantly reduced in neurons of NSE-IκBα-SR mice compared with wild-type littermates (FIG. 4C).

### Example 5

### TWEAK Mediates Apoptosis In Vivo

To investigate whether TWEAK-induced neurodegeneration is relevant in vivo, mice were injected intraperitoneally with the neutralizing anti-TWEAK antibody AB.G11 or an unspecific hamster Ig (200 μg per mouse). The mice were then subjected to distal MCAO. Intraperitoneal injection is an effective administration mode for neutralizing antibodies in cerebral ischemia (van Bruggen et al. (1999) J. Clin. Invest. 104:1613-1620; Martin-Villalba et al. (2001) Cell Death Differ. 8:679-686). The anti-TWEAK antibody had no effect on the various physiological parameters measured (Table 1); however, it significantly reduced the infarct size after 2 days (FIG. 5). This suggests that TWEAK also induces neurodegeneration in vivo.

Table 1: Physiologic parameters immediately before and 15 minutes after MCAO in mice that were i.p. injected with either 200 μg unspecific hamster immunoglobulin or with 200 μg hamster anti-TWEAK AB.G11. None of the parameters differed significantly between the groups (t-Test). Values are means±SEM, n=7-8. MABP, mean arterial blood pressure. Hb, hemoglobin concentration.

TABLE 1

| | Control Antibody | | Anti-TWEAK AB.G11 | |
|---|---|---|---|---|
| Parameter | Pre-MCAO | Post-MCAO | Pre-MCAO | Post-MCAO |
| MABP | 60.0 ± 2.7 | 51.7 ± 2.1 | 61.3 ± 2.3 | 51.3 ± 3.1 |
| (mm Hg) | | | | |
| Heart rate (per | 368.7 ± 7.4 | 391.0 ± 13.3 | 371.5 ± 9.5 | 390.8 ± 12.9 |
| min) | | | | |
| Glucose | 254.6 ± 18.7 | 284.1 ± 33.6 | 231.6 ± 14.2 | 285.3 ± 22.0 |
| (mg/dl) | | | | |
| Arterial pCO₂ | 59.6 ± 4.0 | 65.2 ± 3.3 | 59.2 ± 1.8 | 62.0 ± 1.3 |
| (mm Hg) | | | | |
| Arterial pO₂ | 92.5 ± 10.3 | 98.0 ± 6.3 | 80.5 ± 6.9 | 85.7 ± 4.9 |
| (mm Hg) | | | | |
| Hb (g/l) | 14.9 ± 0.2 | 13.6 ± 0.3 | 15.0 ± 0.2 | 13.9 ± 0.2 |
| Laser doppler | 84.2 ± 5.1 | 16.4 ± 1.9 | 84.6 ± 4.5 | 13.8 ± 1.4 |
| (Relative | | | | |
| Units) | | | | |
| Body weight | 23.8 ± 0.6 | | 23.5 ± 0.4 | |
| (g) | | | | |

### Example 6

### Anti-TWEAK Antibody Decreases Glial Cell Activation

Neuroglial cells (also known as glial cells), such as astrocytes, microglia, and oligodendrocytes, contribute to the inflammatory processes in the nervous system. Upon insult, these cells can be activated and release cytokines (e.g., IL-6 and IL-8), thereby amplifying an inflammatory response. The release of such factors may cause cytotoxicity and contribute to the cell death seen after ischemic injury and stroke.

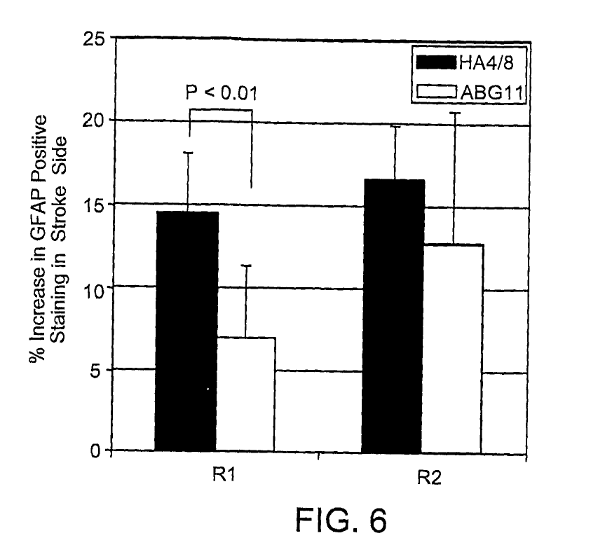

To determine if the TWEAK/TWEAK-R pathway plays a role in glial cell activation and if a TWEAK/TWEAK-R blocking agent could inhibit glial cell activation, the MCAO model of cerebral ischemia in mice was used as described in Example 1. Anti-TWEAK AB.G11 antibody or an unspecific control (HA4/8) was injected intraperitoneally 10 minutes before MCAO. Forty-eight hours after MCAO, mice were deeply re-anesthetized. Coronal brain sections were prepared and used for immunohistochemistry with anti-GFAP (glial fibrillary acidic protein) antibody; with GFAP levels serving as a marker for astrocyte activation. Samples from each treatment (anti-TWEAK and HA4/8) were stained for GFAP. The amount of GFAP-positive staining was quantitated and compared. The results are shown in FIG. 6. Treatment of mice with anti-TWEAK antibody AB.G11 prior to MCAO resulted in a significant reduction in the amount of astrocyte activation in ischemic side, as measured by GFAP-positive staining.

### Example 7

An exemplary sequence of a human TWEAK protein is as follows:

| | |
|---|---|
| MAARRSQRRR GRRGEPGTAL LVPLALGLGL ALACLGLLLA VVSLGSRASL | (SEQ ID NO: 1) |
| SAQEPAQEEL VAEEDQDPSE LNPQTEESQD PAPFLNRLVR PRRSAPKGRK | |
| TRARRAIAAH YEVHPRPGQD GAQAGVDGTV SGWEEARINS SSPLRYNRQI | |
| GEFIVTRAGL YYLYCQVHFD EGKAVYLKLD LLVDGVLALR CLEEFSATAA | |
| SSLGPQLRLC QVSGLLALRP GSSLRIRTLP WAHLKAAPFL TYFGLFQVH | |

An exemplary sequence of a human Fn14 protein is as follows:

| | |
|---|---|
| MARGSLRRLL RLLVLGLWLA LLRSVAGEQA PGTAPCSRGS SWSADLDKCM | (SEQ ID NO: 2) |
| DCASCRARPH SDFCLGCAAA PPAPFRLLWP ILGGALSLTF VLGLLSGFLV | |
| WRRCRRREKF TTPIEETGGE GCPAVALIQ | |

A number of embodiments of the invention have been described. Nevertheless, it will be understood that various modifications may be made without departing from the spirit and scope of the invention. Accordingly, other embodiments are within the scope of the following claims.

1. A method for treating a human subject who has sustained a stroke, the method comprising:
administering, to the subject, an agent that blocks a TWEAK/TWEAK-R interaction or activity, wherein the agent is an antibody or a soluble form of a TWEAK receptor.

2. The method of claim 1 wherein the agent reduces the ability of TWEAK to bind to Fn14.

3. The method of claim 1 wherein the agent is an antibody that binds to TWEAK.

4. The method of claim 1 wherein the agent is an antibody that binds to Fn14.

5. The method of claim 3 wherein the agent is a full length IgG.

6. The method of claim 3 wherein the agent is an antibody that consists of an antigen-binding fragment of a full length IgG.

7. The method of claim 3 wherein the agent is a single chain antibody, Fab fragment, F(ab′)₂ fragment, Fd fragment, Fv fragment, or dAb fragment.

8. The method of claim 3 wherein the agent is a human or humanized antibody or antigen-binding fragment thereof.

9. The method of claim 1 wherein the agent is a soluble form of a TWEAK receptor.

10. The method of claim 9 wherein the soluble form of the TWEAK receptor is fused with an antibody Fc region.

11. The method of claim 9, wherein the soluble form of the TWEAK receptor is at least 95% identical to amino acids 28-X₁ of SEQ ID NO:2, where amino acid X₁ is selected from the group of residues 68 to 80 of SEQ ID NO:2.

12. The method of claim 1 wherein the agent is administered in an amount sufficient to reduce ischemic damage in neuronal tissue in the brain.

13. The method of claim 1 wherein the agent is administered in an amount sufficient to reduce infarct size in neuronal tissue in the brain, relative to a similarly affected, but untreated subject.

14. The method of claim 1 wherein the stroke is hemorrhagic stroke.

15. The method of claim 1 wherein the stroke is ischemic stroke.

16. The method of claim 1 wherein the stroke is a transient ischemic attack.

17. The method of claim 1 wherein the subject has experienced a stroke within the previous 48 hours.

18. The method of claim 1 wherein the agent is administered in combination with another treatment for stroke.

19. The method of claim 16 wherein the other treatment for stroke comprises administering a thrombolytic agent.

20. The method of claim 1 further comprising: evaluating the subject using a stroke assessment criterion.

21. A method comprising:
identifying a subject who has had a stroke; and
administering to the subject an agent that blocks a TWEAK/TWEAK-R interaction or activity.

22. A method comprising:
detecting a recent stroke event in a subject; and
administering to the subject an agent that blocks a TWEAK/TWEAK-R interaction or activity.

23. A container comprising:
an agent that blocks a TWEAK/TWEAK-R interaction or activity; and
a label with instructions for use of the agent in treating stroke.

24. A method of evaluating a subject for a stroke- or stroke-risk assessment, the method comprising evaluating a TWEAK or TWEAK receptor protein or a nucleic acid encoding a TWEAK or TWEAK receptor in the subject or in a sample obtained from the subject.

25-30. (canceled)

31. A method of monitoring efficacy of a treatment for stroke, the method comprising:
treating a subject for a stroke; and
evaluating a TWEAK or TWEAK receptor protein or a nucleic acid encoding a TWEAK or TWEAK receptor in the subject or in a sample obtained from the subject.

32. A method of identifying a subject for stroke treatment, the method comprising:
evaluating a TWEAK or TWEAK receptor protein or a nucleic acid encoding a TWEAK or TWEAK receptor in the subject or in a sample obtained from the subject; and
identifying the subject as a subject suited for stroke treatment as a function of results of the evaluating.

33. A method of selecting a patient population for treatment, the method comprising:
evaluating expression of a TWEAK or TWEAK receptor in one or more subjects; and
selecting a set of one or more subjects who have elevated expression of a TWEAK or TWEAK receptor relative to a reference.

34-36. (canceled)

37. The method of claim 4 wherein the agent is a full length IgG.

38. The method of claim 4 wherein the agent is an antibody that consists of an antigen-binding fragment of a full length IgG.

39. The method of claim 4 wherein the agent is a single chain antibody, Fab fragment, F(ab′)₂ fragment, Fd fragment, Fv fragment, or dAb fragment.

40. The method of claim 4 wherein the agent is a human or humanized antibody or antigen-binding fragment thereof.
